(19) United States
(12) Patent Application Publication
(10) Pub. No.: US 2010/0168243 A1
(43) Pub. Date: Jul. 1, 2010

(54) Renin Inhibitors

(76) Inventors: John J. Baldwin, Gwynedd Valley, PA (US); Salvacion Cacatian, Blue Bell, PA (US); David A. Claremon, Maple Glen, PA (US); Lawrence W. Dillard, Yardley, PA (US); Patrick T. Flaherty, Pittsburgh, PA (US); Alexey V. Ishehenko, Sornerville, MA (US); Lanqi Jia, Horsham, PA (US); Gerard McGeehan, Garnet Valley, PA (US); Robert D. Simpson, Wilmington, DE (US); Suresh B. Singh, Kendall Park, NJ (US); Colin M. Tice, Ambler, PA (US); Zhenrong Xu, Horsham, PA (US); Jing Yuan, Lansdale, PA (US); Wei Zhao, Eagleville, PA (US); Linghang Zhuang, Chalfont, PA (US); Jing Zhang, Waltham, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/665,222

(22) PCT Filed: Jun. 20, 2008
(86) PCT No.: PCT/US08/07704

§ 371 Date: Feb. 5, 2010

Related U.S. Application Data
(60) Provisional application No. 60/936,380, filed on Jun. 20, 2007.

(51) Int. Cl. A61K 31/166; C07C 237/24; A61K 31/165; C07C 237/32; A61P 9/00; A61P 7/10; A61P 5/40; A61P 9/12

(52) U.S. Cl. 514/620; 564/189; 514/613; 564/164; 564/157

(57) ABSTRACT

The present invention is directed to aspartic protease inhibitors. The present invention is also directed to pharmaceutical compositions comprising the disclosed aspartic protease inhibitors. The present invention is further directed to methods of antagonizing one or more aspartic proteases in a subject in need thereof, and methods for treating an aspartic protease mediated disorder in a subject using the disclosed aspartic protease inhibitors.

### RELATED APPLICATION

This application claims the benefit of U.S. Provisional Application No. 60/936,380, filed Jun. 20, 2007. The entire teachings of the above application are incorporated herein by reference.

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin 1, the product of renin catalyzed cleavage of angiotensinogen are present. Elevated levels of β-amyloid, the product of BACE activity on amyloid precursor protein, are widely believed to be responsible for the amyloid plaques present in the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of ATI receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001, 56, 21). The present invention relates to the unexpected identification of renin inhibitors of a non-peptidic nature and of low molecular weight. Orally active renin inhibitors, which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference by its entirety.

### SUMMARY OF THE INVENTION

One embodiment of the present invention is aspartic protease inhibitor represented by Structural Formula (I):

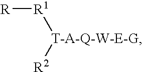

or an enantiomer, a diastereomer or a pharmaceutically acceptable salt thereof,
wherein:

### R is:

a) hydrogen;
b) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cycloalkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino, or tri(C₁-C₄)alkylsilyl, each optionally and independently substituted with zero to four substituents selected from the group consisting of halogen, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;
c) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃)alkenyl, or heteroaryl(C₂-C₃)alkynyl, each optionally and independently substituted with zero to three substituents selected from the group consisting of: halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or
d) a divalent radical selected from —(CH₂)₃—, —(CH₂)₄—, —(CH₂)₅— or —(CH₂)₆—, which is attached to R¹ to form a fused or spirofused ring system, and is optionally and independently substituted with zero to four substituents selected from: halogen, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally and independently substituted with zero to four substituents selected from: halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl;

### R² is:

### a) —H; or

b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxycarbonyl(C₁-C₆)alkyl amino, (C₁-C₆)acyloxy(C₁-C₆)alkyl, (C₁-C₆) acyloxy(C₁-C₆)alkoxy, (C₁-C₆) acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkyl, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonylamino(C₁-C₆)alkyl, (C₁-C₆)alkylaminocarbonylamino(C₁-C₆)alkoxy, (C₁-C₆)alkylaminocarbonylamino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)allylthio, (C₁-C₆)alkylaminocarboxy(C₁-C₆)alkylamino, (C₁-C₁₂)alkoxycarbonylamino, (C₁-C₁₂)alkylaminocarbonylamino, or (C₁-C₁₂)alkanoylamino, each optionally substituted by:
1) 1 to 5 halogen atoms; and/or
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
wherein the divalent sulfur atoms are optionally and independently oxidized to sulfoxide or sulfone, and wherein the carbonyl groups are optionally and independently changed to a thiocarbonyl groups;

### T is N or CR³;

R³ is hydrogen, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonylamino, phenylamino or heteroarylamino in which each phenylamino or heteroarylamino group is optionally substituted with 1 to 5 groups independently selected from the group consisting of halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl;

provided that:

i) R² and R³ are not both hydrogen; and
ii) when T is N or T is CR³ and R³ is hydroxy, halogen, or optionally substituted phenylamino or heteroarylamino, R² is not an optionally substituted alkoxy, alkylthio or amino group as follows: (C₁-C₁₂)alkoxy, (C₁-C₁₂)alkylthio, (C₁-C₁₂)allylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)allylthio, (C₁-C₆)alkylamino(C₁-C₆)alkylamino, aminocarbonylamino(C₁-C₁₂)alkoxy, aminocarbonylamino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl(C₁-C₆)alkylamino, (C₁-C₆) acyloxy(C₁-C₆)alkoxy, (C₁-C₆) acyloxy(C₁-C₆)alkylthio, (C₁-C₆)acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonylamino(C₁-C₆)alkoxy, (C₁-C₆)alkylaminocarbonylamino(C₁-C₆)alkylthio, (C₁-C₆)alkylaminocarbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylaminocarbonylamino, or (C₁-C₁₂)alkanoylamino, each optionally substituted by:
1) 1 to 5 halogen atoms; and/or
2) 1 group selected from cyano, hydroxy, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, or halo(C₃-C₆)cycloalkoxy;
wherein the divalent sulfur atoms are optionally and independently oxidized to sulfoxide or sulfone, and wherein the carbonyl groups are optionally and independently changed to thiocarbonyl groups;
R^{a} and R^{b}, for each occurrence, are independently hydrogen, (C₁-C₆)alkyl, or halo(C₁-C₆)alkyl or R^{a} and R^{b} attached to one carbon atom taken together are an oxo;
A is represented by the following Structural Formula:

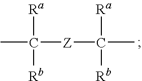

Z is —O—, —S—, —(NR⁷)— or —(CR^{a}R^{b})—, wherein R⁷ is (C₁-C₆)alkyl, halo(C₁-C₆)alkyl or (C₁-C₃)alkoxy(C₁-C₃)alkyl;
m and n are 0, 1 or 2 and m+n=2; provided when T is N and Z is —O—, —S— or —NR⁷—, then n is 2;
Q is a divalent radical selected from

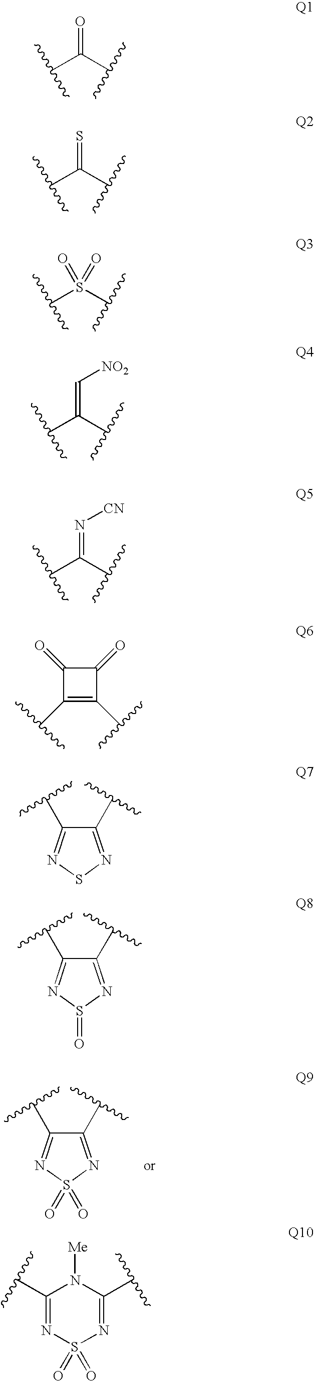

wherein A and W or E are attached to the truncated bonds
W is a bond or a (C₁-C₆) alkylene; and
W is optionally and independently substituted by zero to four groups selected from:

1) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, saturated heterocyclyl(C₁-C₃)alkyl, hydroxy and oxo, wherein each of these groups is optionally and independently substituted by zero to six groups selected from: halogen, cyano, hydroxy, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy and wherein divalent sulfur atoms are optionally oxidized to sulfoxide or sulfone; or

2) phenyl, naphthyl, heteroaryl, phenyl(C₁-C₃)alkyl, naphthyl(C₁-C₃)alkyl, and heteroaryl(C₁-C₃)alkyl, each optionally and independently substituted with zero to three groups selected from: halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃, C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈) cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)allyl, (C₁-C₈)alkoxycarbonyl(C₁-C₆)alkyl, (C₁-C₈)acyloxy(C₁-C₆)alkyl, aminocarbonyl(C₁-C₆)allyl, (C₁-C₈)alkylaminocarbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally and independently substituted with zero to three groups selected from: halogen, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;

E is a saturated or unsaturated 3-, 4-, 5-, 6-, or 7-membered ring which is optionally bridged by (CH₂)ₙ via bonds to two members of said ring, wherein said ring is composed of carbon atoms and zero to four hetero atoms selected from: zero to four nitrogen atoms, zero or one oxygen atoms, and zero or one sulfur atoms, said ring being optionally and independently substituted with zero to four groups selected from: halogen, hydroxy, (C₁-C₆)alkyl, (C₃-C₈)cycloalkyl(C₁-C₆)alkyl, halo(C₁-C₆)alkyl, hydroxy(C₁-C₆)alkyl, and oxo groups, such that when there is substitution with one oxo group on a carbon atom it forms a carbonyl group, and when there is substitution of one or two oxo groups on sulfur it forms sulfoxide or sulfone groups, respectively;
n is 1 to 3;
G is hydrogen, (C₁-C₆)alkyl, (C₄-C₇)heterocyclyl, hydroxy, hydroxy(C₁-C₆)alkyl, —NR^{4a}R⁴, —O(C₁-C₆)alkyl-NR^{4a}R⁴, amino(C₁-C₆)alkylcarboxy, (C₃-C₈)cycloalkyl, (C₁-C₆)alkylamino(C₁-C₆)alkyl, amino(C₁-C₆)alkyl, (C₁-C₆)alkylamino, di(C₁-C₆)alkylamino, di(C₁-C₆)alkylamino(C₁-C₆)alkyl, C(═NH)NH₂, C(═NH)NHR⁴, NHC(═NH)NH₂, NHC(═NH)NHR⁴; —(C₀-C₆)alkyl-NR⁴R^{4a}, —NHC(═NH)NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino;

and where R^{4a} is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, amino, (C₁-C₆)alkyl, (C₁-C₆)alkylamino, halo(C₃-C₆)alkyl, hydroxy(C₁-C₆)alkyl, amino(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;

provided that when T is N, E is not 1,2-cyclopentylene.

Another embodiment of the invention is a pharmaceutical composition comprising a pharmaceutically acceptable carrier or diluent and an aspartic protease inhibitor disclosed herein (e.g., a compound represented by Structural Formula (I) or an enantiomer, a diastereomer or a pharmaceutically acceptable salt thereof). The pharmaceutical composition is used in therapy, e.g., for inhibiting an aspartic protease mediated disorder in a subject.

Another embodiment of the invention is a method of antagonizing one or more aspartic proteases in a subject in need of such treatment. The method comprises administering to the subject an effective amount of an aspartic protease inhibitor disclosed herein (e.g., a compound represented by Structural Formula (I) or an enantiomer, a diastereomer or a pharmaceutically acceptable salt thereof).

Another embodiment of the invention is a method of treating or ameliorating an aspartic protease mediated disorder in a subject. The method comprises administering to the subject an effective amount of an aspartic protease inhibitor disclosed herein (e.g., a compound represented by Structural Formula (I) or an enantiomer, a diastereomer or a pharmaceutically acceptable salt thereof).

Another embodiment of the invention is a method of treating or ameliorating a renin mediated disorder mediated disorder in a subject. The method comprises administering to the subject an effective amount of an aspartic protease inhibitor disclosed herein (e.g., a compound represented by Structural Formula (I) or an enantiomer, a diastereomer or a pharmaceutically acceptable salt thereof).

Another embodiment of the invention is a method of treating hypertension in a subject. The method comprises administering to the subject an effective amount of an aspartic protease inhibitor disclosed herein (e.g., a compound represented by Structural Formula (I) or an enantiomer, a diastereomer or a pharmaceutically acceptable salt thereof).

Another embodiment of the invention is the use of an aspartic protease inhibitor disclosed herein (e.g., a compound represented by Structural Formula (I) or an enantiomer, a diastereomer or a pharmaceutically acceptable salt thereof)) for the manufacture of a medicament for antagonizing one or more aspartic proteases in a subject in need of such treatment.

Another embodiment of the invention is the use of an aspartic protease inhibitor disclosed herein (e.g., a compound represented by Structural Formula (I) or an enantiomer, a diastereomer or a pharmaceutically acceptable salt thereof)) for the manufacture of a medicament for treating or ameliorating an aspartic protease mediated disorder in a subject.

Another embodiment of the invention is the use of an aspartic protease inhibitor disclosed herein (e.g., a compound represented by Structural Formula (I) or an enantiomer, a diastereomer or a pharmaceutically acceptable salt thereof)) for the manufacture of a medicament for treating or ameliorating the renin mediated disorder in a subject.

Another embodiment of the invention is the use of an aspartic protease inhibitor disclosed herein (e.g., a compound represented by Structural Formula (I) or an enantiomer, a diastereomer or a pharmaceutically acceptable salt thereof)) for the manufacture of a medicament for treating hypertension in a subject.

### DETAILED DESCRIPTION OF THE INVENTION

The present invention is directed to an aspartic protease inhibitor represented by Structural Formula (I) or an enantiomer, a diastereomer or a pharmaceutically acceptable salt thereof. Values and particular values for the variables in Structural Formula (I) are provided in the following paragraphs. For Structural Formula I:

R is a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: halogen, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or c) a divalent radical selected from —(CH₂)₄— or —(CH₂)₅—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo.

In a particular embodiment, R is a) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from the group consisting of fluorine, hydroxy, (C₁-C₃)alkyl, and halo(C₁-C₃)alkyl,

b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from the group consisting of halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or c) a divalent radical selected from —(CH₂)₄— or —(CH₂)₅—, which is attached to R¹ to form a fused or spirofused ring system.

In another particular embodiment, R is a) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, or halo(C₁-C₃)alkyl; or b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to 3′ substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio or H₂NCO; or

c) —(CH₂)₄— or —(CH₂)₅—, which is attached to R¹ to form a fused or spirofused ring system.

In another particular embodiment, R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—, which is attached to R¹ to form a fused or spirofused ring system.

In another particular embodiment, R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl.

In another particular embodiment, R is phenyl, 3-methylphenyl or 3-ethylphenyl.

R¹ is phenyl, monocyclic heteroaryl, bicyclic heteroaryl, benzo-1,3-dioxole, or (C₃-C₇)cycloalkyl ring optionally substituted with up to four substituents independently selected from the group consisting of fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl.

In a particular embodiment, R¹ is phenyl, monocyclic heteroaryl ring, bicyclic heteroaryl ring or benzo-1,3-dioxole, optionally substituted with up to four substituents independently selected from the group consisting of halogen, cyano, (C₁-C₃)alkyl, (C₃-C₄)cycloalkyl, halo(C₁-C₃)alkyl, —C₃)alkoxy, halo(C₁-C₃)alkoxy, and H₂NCO.

In another particular embodiment, R¹ is phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—.

In another particular embodiment, R¹ is phenyl optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—.

In another particular embodiment, R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole.

In another particular embodiment, R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl.

In another particular embodiment, R¹ is phenyl or 3-chlorophenyl.

R² is a)—H; or b) (C₁-C₁₀)alkyl, (C₂-C₁₀)alkenyl, (C₂-C₁₀)alkynyl, (C₁-C₁₀)alkoxy, (C₁-Cₒ)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkyl, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted by

1) 1 to 5 fluorine atoms; and/or
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy; wherein the divalent sulfur atoms are independently optionally oxidized to sulfoxide or sulfone.

In a particular embodiment, R² is —H, (C₁-C₈)alkyl, (C₄-C₉)cycloalkylalkyl, fluoro(C₁-C₈)alkyl, fluoro(C₄-C₉)-cycloalkylalkyl, (C₁-C₈)alkoxy, (C₄-C₉)cycloalkylalkoxy, fluoro(C₁-C₈)alkoxy, hydroxy(C₁-C₈)alkyl, (Cᵣ C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)-cycloalkanecarbonyllamino(C₁-C₅)alkyl, (C₃-C₄)cycloalkanecarbonyllamino(C₁-C₅)alkoxy, aminosulfonylamino(C₁-C₈)alkyl, aminosulfonylamino(C₁-C₈)alkoxy, (C₁-C₅)alkane-sulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylamino-carbonylamino(C₁-C₅)alkyl, C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino.

In a particular embodiment, R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₄)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl, (C₁-C₃)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy.

In another particular embodiment, R² is (C₁-C₃)alkoxy(C₁-C₅)alkyl, (C₁-C₃)alkoxy(C₁-C₅)alkoxy, (C₁-C₃)alkoxycarbonylamino(C₁-C₅)alkyl, (C₁-C₃)-alkoxycarbonylamino(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, or (C₁-C₅)alkylamino-carboxy(C₁-C₅)alkoxy. More specifically, R² is 4-methoxybutyl, 4-ethoxybutyl, 4-methoxypentyl, 3-methoxypropoxy, 3-(methoxycarbonylamino)propyl, or 2-(methoxycarbonylamino)ethoxy.

In another particular embodiment, R² is 3-(acetylamino)propyl or 2-(acetylamino)ethoxy.

R³ is —H, halogen, (C₁-C₃)alkyl, (C₁-C₃)alkoxy, hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylaminocarbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from the group consisting of fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;

provided that
i) R² and R³ are not both hydrogen and
ii) when T is N or T is CR³ and R³ is hydroxyl, halogen, or optionally substituted phenylamino or heteroarylamino, R² is not an optionally substituted alkoxy, alkylthio or amino group as follows: (C₁-C₁₀)alkoxy, (C₁-C₁₀)alkylthio, (C₁-C₁₀)alkylamino, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, aminocarbonylamino(C₁-C₁₀)alkoxy, aminocarbonylamino(C₁-C₁₀)alkylthio, aminocarbonyl-amino(C₁-C₁₀)alkylamino, (C₁-C₅)alkanoylamino(C₁-C₈)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted with

1) 1 to 5 fluorine atoms; and/or

2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy;

wherein the divalent sulfur atoms are independently optionally oxidized to sulfoxide or sulfone.

In a particular embodiment, R³ is —H, halogen, OH, (C₁-C₄)alkanoylamino, or (C₁-C₃)alkoxy;

provided that

i) R² and R³ are not both hydrogen; and
ii) when T is N or T is CR³ and R³ is OH or halogen, R² is not an optionally substituted alkoxy, alkylthio or amino group as follows: (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)-alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkanecarbonyllamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxy-carbonylamino, (C₁-C₈)alkylaminocarbonylamino, (C₁-C₈)alkanoylamino, fluoro(C₁-C₈)alkoxy-carbonylamino, fluoro(C₁-C₈)alkylaminocarbonylamino, or fluoro(C₁-C₈)alkanoylamino.

In another particular embodiment, R³ is hydrogen, fluoro, hydroxyl, or (C₁-C₄)alkanoylamino, provided that when T is N or T is CR³ and R³ is hydroxyl or fluoro, R² is not (C₁-C₃)alkoxy(C₁-C₅)alkoxy, (C₃-C₄)cycloalkyl(C₁-C₅)alkoxy, (C₁-C₃)alkoxy-carbonylamino(C₁-C₅)alkoxy, (C₁-C₃)alkanoylamino(C₁-C₅)alkoxy or (C₁-C₃)alkylaminocarbonyl(C₁-C₅)alkoxy.

In another particular embodiment, R³ is hydrogen, hydroxyl or methoxycarbonylamino, provided that when R³ is hydroxyl, R² is not 3-methoxypropoxy, 2-(acetylamino)ethoxy, or 2-(methoxycarbonylamino)ethoxy. More specifically, R³ is hydroxyl.

Q is Q1 or Q2. More specifically, Q is Q1.

W is a bond or a (C₁-C₃)alkyl. In a particular embodiment, W is a bond.

E is a saturated or unsaturated 3-, 4-, 5-, 6-, or 7-membered ring which is optionally bridged by (CH₂)ₙ via bonds to two members of said ring, wherein said rind is composed of carbon atoms and zero to four hetero atoms selected from: zero to four nitrogen atoms, zero or one oxygen atoms, and zero or one sulfur atoms, said ring being optionally and independently substituted with zero to four groups selected from: halogen, hydroxy, (C₁-C₆)alkyl, (C₃-C₈)cycloalkyl(C₁-C₆)alkyl, halo(C₁-C₆)alkyl, hydroxy(C₁-C₆)alkyl, and oxo groups, such that when there is substitution with one oxo group on a carbon atom it forms a carbonyl group, and when there is substitution of one or two oxo groups on sulfur it forms sulfoxide or sulfone groups, respectively; n is 1 to 3.

In a particular embodiment, E is a saturated 4-, 5-, 6-, or 7-membered heterocyclic ring which is optionally bridged by (CH₂)_{q} via bonds to two members of said ring; wherein E is optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively. More specifically, the heterocyclic ring is represented by the following Structural Formula:

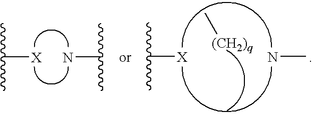

In another particular embodiment, E is represented by the following Structural Formula:

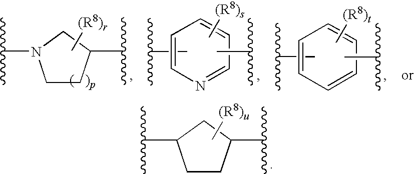

In another particular embodiment, when T is N, E is not 1,2-cyclopentylene.

G is hydrogen, (C₁-C₆)alkyl, (C₄-C₇)heterocyclyl, hydroxy, hydroxy(C₁-C₆)alkyl, —NR^{4a}R⁴, —O(C₁-C₆)alkyl-NR^{4a}R⁴, amino(C₁-C₆)alkylcarboxy, (C₃-C₈)cycloalkyl, (C₁-C₆)allylamino(C₁-C₆)alkyl, amino(C₁-C₆)alkyl, C₆)alkylamino, di(C₁-C₆)alkylamino, di(C₁-C₆)alkylamino(C₁-C₆)alkyl, C(═NH)NH₂, C(═NH)NHR⁴, NHC(═NH)NH₂, NHC(═NH)NHR⁴; —(C₀-C₆)alkyl-NR⁴R^{4a}, —NHC(═NH)NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino;

and where R^{4a} is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively.

In a particular embodiment, G is hydrogen, (C₁-C₆)alkyl, heterocyclyl, —(C₂-C₆)alkyl-OH, —(C₂-C₆)alkyl-NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino.

In another particular embodiment, G is hydrogen, heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkyl aryl, —C(═O)(C₁-C₄)alkyl(C₄-C₇)heterocyclyl, —(C₁-C₄)alkyl(C₃-C₇)cycloalkyl, or —(C₁-C₄)alkyl(C₄-C₇)heterocyclyl.

In another particular embodiment, G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl.

In another particular embodiment, G is hydroxy, —NR⁴R^{4a}, —O(C₂-C₆)alkyl-NR⁴R^{4a}, heterocyclyl, —(C₁-C₆)alkyl-OH, —(C₁-C₆)alkyl-NR⁴R^{4a}, —C(═O)(C₁-C₆)alkyl-NR⁴R^{4a}, —C(═NH)NR⁴R^{4a}, —NHC(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino.

In another particular embodiment, G is —NHR⁹ or (C₁-C₃)alkyl-NHR⁹. More specifically, G is —NH₂ or —CH₂NH₂.

In a 1^{st} specific embodiment, the aspartic protease inhibitor of the present invention is represented by a Structural Formula selected from (Ia), (Ia′), (Ib), (Ic), (Id), (Ie), (If), (Ig), (Ih), (Ii), (Ij), (Ik) and (Il):

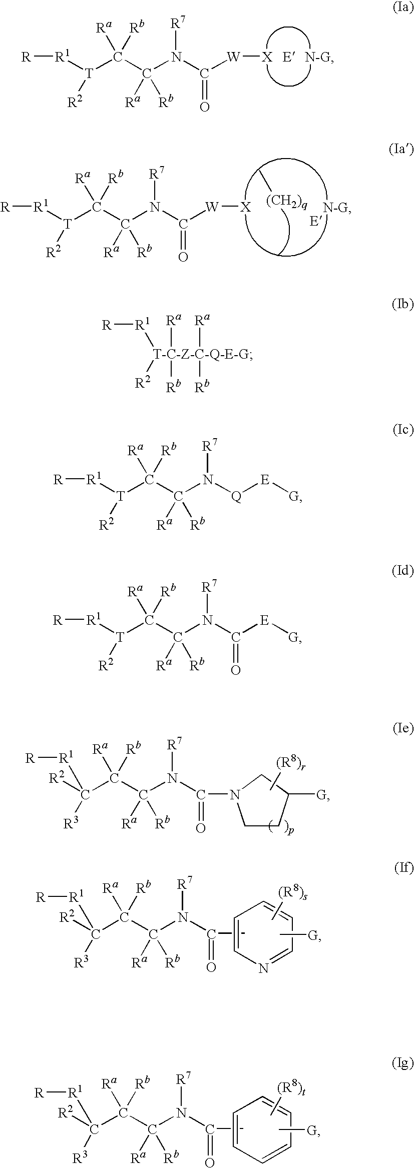

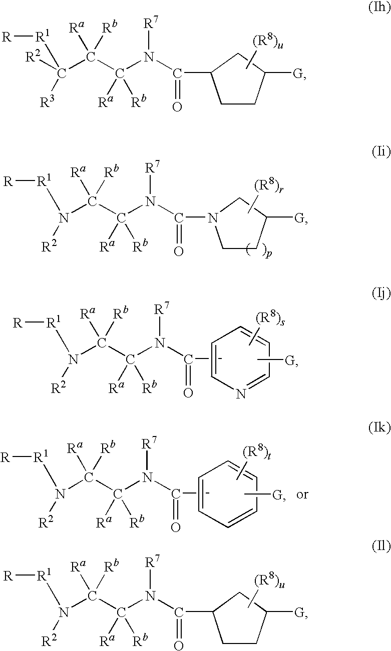

or an enantiomer, a diastereomer or a pharmaceutically acceptable salt thereof, wherein:

ring E′ is a saturated 4-, 5-, 6-, or 7-membered heterocyclic ring which is optionally bridged by (CH₂)_{q} via bonds to two members of said ring; wherein ring E′ is optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;

X is a ring carbon atom or nitrogen atom bonded directed to W;

W is a bond or a (C₁-C₃)alkyl;

q is 1 to 3;

R⁸ is selected from the group consisting of halogen, hydroxy, (C₁-C₆)alkyl, halo(C₁-C₆)alkyl, hydroxy(C₁-C₆)alkyl and oxo groups such that when there is substitution with one oxo group on a carbon atom it forms a carbonyl group;

p is 1 or 2

r is 0, 1 or 2 when p is 1 or r is 0, 1, 2 or 3 when p is 2;

s is 0, 1, 2, 3 or 4;

t is 0, 1, 2, 3 or 4;

u is 0, 1, 2 or 3; and

the values and specific values for the remainder of the variables are as described above for Structural Formula (I).

In a more specific embodiment, ring E′ in Structural Formula (Ia) is selected from the group consisting of piperidinyl, piperazinyl, and pyrrolidinyl, said group being optionally substituted with a hydroxy, (C₁-C₃)alkyl or halo(C₁-C₃)alkyl group. Values and specific values for the remainder of the variables are as described above for Structural Formula (I).

In another more specific embodiment, for Structural Formula (Ia), ring E′ is selected from the group consisting of piperidinyl, piperazinyl, and pyrrolidinyl, said group being optionally substituted with a hydroxy, (C₁-C₃)alkyl or halo(C₁-C₃)alkyl group; and G is hydrogen, (C₁-C₆)alkyl, heterocyclyl, —(C₂-C₆)alkyl-OH, —(C₂-C₆)alkyl-NR⁴R^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino. Even more specifically, G is hydrogen, heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₇)cycloalkyl, or —(C₁-C₄)alkyl(C₄-C₇)heterocyclyl. Values and specific values for the remainder of the variables are as described above for Structural Formula (I).

In another more specific embodiment, for Structural Formula (Ie), (If), (Ih), (Ii), (Ij) or (Il), G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)allyl, C(═NH)NH₂, C(═NH)NHR⁴, NHC(═NH)NH₂, or NHC(═NH)NHR⁴; wherein R⁴ is (C₁-C₃)alkyl. Values and specific values for the remainder of the variables are as described above for Structural Formula (I).

In another more specific embodiment, G in Structural Formula (Ig) or (Ik) is hydroxy, —NR⁴R^{4a}, —O(C₂-C₆)alkyl-NR⁴R^{4a}, heterocyclyl, —(C₁-C₆)alkyl-OH, —(C₁-C₆)alkyl-NR⁴R^{4a}, —C(═O)(C₁-C₆)alkyl-NR⁴R^{4a}, —C(═NH)NR⁴R^{4a}, —NHC(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino. Values and specific values for the remainder of the variables are as described above for Structural Formula (I).

In a 2^{nd} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik), or (Il) or an enantiomer, a diastereomer or a pharmaceutically acceptable salt thereof, wherein:

R is a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈) alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: halogen, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or c) a divalent radical selected from —(CH₂)₄— or —(CH₂)₅—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;

R¹ is phenyl, monocyclic heteroaryl, bicyclic heteroaryl, benzo-1,3-dioxole, or (C₃-C₇)cycloalkyl ring optionally substituted with up to four substituents independently selected from the group consisting of fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl;

R² for Structural Formulas (Ia), (Ia′), (Ib), (Ic), (Id), (Ie), (If), (Ig), and (Ih) is

a) —H; or b) (C₁-C₁₀)alkyl, (C₂-C₁₀)alkenyl, (C₂-C₁₀)alkynyl, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)allylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)allylamino, (C₁-C₅)alkoxycarbonylamino(C₁-C₅)alkyl, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkyl, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted by
1) 1 to 5 fluorine atoms; and/or
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy; wherein the divalent sulfur atoms are independently optionally oxidized to sulfoxide or sulfone;

R² for Structural Formulas (Ii), (Ik) and (Il) is a) —H; or b) (C₁-C₁₀)alkyl, (C₂-C₁₀)alkenyl, (C₂-C₁₀)alkynyl, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₃)alkoxy(C₁-C₃)alkoxy(C₁-C₃)alkyl, aminocarbonylamino(C₁-C₁₀)alkyl, (C₁-C₅)alkanoylamino(C₁-C₅)alkyl, aminosulfonylamino(C₁-C₁₀)alkyl, (C₁-C₅)alkanesulfonylamino(C₁-C₅)alkyl, formylamino(C₁-C₅)alkyl, (C₁-C₅)alkoxycarbonylamino(C₁-C₅)alkyl, (C₁-C₅)alkylaminocarbonylamino(C₁-C₅)alkyl, aminocarbonyl(C₁-C₅)alkyl, (C₁-C₅)alkylaminocarbonyl(C₁-C₅)alkyl, aminocarboxy(C₁-C₅)alkyl, (C₁-C₅)alkylaminocarboxy(C₁-C₅)alkyl, each optionally substituted by

1) 1 to 5 fluorine atoms; and/or
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy; wherein the divalent sulfur atoms are independently optionally oxidized to sulfoxide or sulfone; and

R³ for Structural Formulas (Ia), (Ia′), (Ib), (Ic), (Id), (Ie), (If), (Ig) and (Ih) is —H, halogen, (C₁-C₃)alkyl, (C₁-C₃)alkoxy, hydroxyl, hydroxy(C₁-C₃)alkyl, hydroxy(C₁-C₃)alkoxy, (C₁-C₄)alkanoylamino, (C₁-C₃)alkoxycarbonylamino, (Cᵣ C₃)alkylaminocarbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from the group consisting of fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;

provided that

iii) R² and R³ are not both hydrogen and
iv) when T is N or T is CR³ and R³ is hydroxyl, halogen, or optionally substituted phenylamino or heteroarylamino, R² is not (C₁-C₁₀)alkoxy, (C₁-C₁₀)alkylthio, (C₁-C₁₀)alkylamino, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)allylamino, aminocarbonylamino(C₁-C₁₀)alkoxy, aminocarbonylamino(C₁-C₁₀)alkylthio, aminocarbonyl-amino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted with

1) 1 to 5 fluorine atoms; and/or

2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy;

wherein the divalent sulfur atoms are independently optionally oxidized to sulfoxide or sulfone.

Values and specific values for the remainder of the variables are as described above in the 1^{st} specific embodiment.

In a 3^{rd} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R is a) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from the group consisting of fluorine, hydroxy, (C₁-C₃)alkyl, and halo(C₁-C₃)alkyl, b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from the group consisting of halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or c) a divalent radical selected from —(CH₂)₄— or —(CH₂)₅—, which is attached to R¹ to form a fused or spirofused ring system;

R¹ is phenyl, monocyclic heteroaryl ring, bicyclic heteroaryl ring or benzo-1,3-dioxole, optionally substituted with up to four substituents independently selected from the group consisting of halogen, cyano, (C₁-C₃)alkyl, (C₃-C₄)cycloalkyl, halo(C₁-C₃)alkyl, (C₁-C₃)alkoxy, halo(C₁-C₃)alkoxy, and H₂NCO;

R² for Structural Formulas (Ia), (Ia′), (Ib), (Ic), (Id), (Ie), (If), (Ig) and (Ih) is —H, (C₁-C₈)alkyl, (C₄-C₉)cycloalkylalkyl, fluoro(C₁-C₈)alkyl, fluoro(C₄-C₉)-cycloalkylalkyl, (C₁-C₈)alkoxy, (C₄-C₉)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)-cycloalkanecarbonyllamino(C₁-C₅)alkyl, (C₃-C₄)cycloalkanecarbonyllamino(C₁-C₅)alkoxy, aminosulfonylamino(C₁-C₈)alkyl, aminosulfonylamino(C₁-C₈)alkoxy, (C₁-C₅)alkane-sulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylamino-carbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;

R² for Structural Formulas (Ii), (Ij), (Ik) and (Il) is R² is —H, (C₁-C₈)alkyl, (C₄-C₉)cycloalkylalkyl, fluoro(C₁-C₈)alkyl, fluoro(C₄-C₉)-cycloalkylalkyl, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, (C₁-C₅)alkanoylamino(C₁-C₅)alkyl, fluoro(C₁-C₅)alkanoylamino(C₁-C₅)alkyl, (C₁-C₃)alkoxy(C₁-C₅)alkanoylamino(C₁-C₅)alkyl, (C₃-C₄)-cycloalkanecarbonyllamino(C₁-C₅)alkyl, aminosulfonylamino(C₁-C₈)alkyl, (C₁-C₅)alkane-sulfonylamino(C₁-C₅)alkyl, formylamino(C₁-C₅)alkyl, (C₁-C₅)alkoxycarbonylamino(C₁-C₅)alkyl, (C₁-C₅)alkylaminocarbonylamino(C₁-C₅)alkyl, (C₁-C₅)alkylamino-carbonylamino(C₁-C₅)alkyl, aminocarbonyl(C₁-C₅)alkyl, aminocarbonyl(C₁-C₅)alkoxy, (C₁-C₅)alkylaminocarbonyl(C₁-C₅)alkyl, aminocarboxy(C₁-C₅)alkyl, or (C₁-C₅)alkylaminocarboxy(C₁-C₅)alkyl;

R³ for Structural Formulas (Ia), (Ia′), (Ib), (Ic), (Id), (Ie), (If), (Ig) and (Ih) is —H, halogen, OH, (C₁-C₄)alkanoylamino, or (C₁-C₃)alkoxy;

provided that

iii) R² and R³ are not both hydrogen; and
iv) when T is N or T is CR³ and R³ is OH or halogen, R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)-alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkanecarbonyllamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxy-carbonylamino, (C₁-C₈)alkylaminocarbonylamino, (C₁-C₈)alkanoylamino, fluoro(C₁-C₈)alkoxy-carbonylamino, fluoro(C₁-C₈)alkylaminocarbonylamino, or fluoro(C₁-C₈)alkanoylamino.

Values and specific values for the remainder of the variables are as described above in the 1^{st} specific embodiment.

In a 4^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R is a) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, or halo(C₁-C₃)alkyl; or

b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio or H₂NCO; or

### c) —(CH₂)₄— or —(CH₂)₅—;

R¹ is phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—;

R² for Structural Formulas (Ia), (Ia′), (Ib), (Ic), (Id), (Ie), (If), (Ig) and (Ih) is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₄)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl, (C₁-C₃)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy;

R² for Structural Formulas (Ii), (Ij), (Ik) and (Il) is (C₁-C₃)alkoxy(C₁-C₅)alkyl, (C₃-C₄)cycloalkyl(C₁-C₅)alkyl, (C₁-C₃)alkoxycarbonylamino(C₁-C₅)alkyl, (C₁-C₃)alkanoylamino(C₁-C₅)alkyl, (C₁-C₃)alkylaminocarbonyl(C₁-C₅)alkyl, aminocarboxy(C₁-C₅)alkyl, or (C₁-C₅)alkylaminocarboxy(C₁-C₅)alkyl

R³ for Structural Formulas (Ia), (Ia′), (Ib), (Ic), (Id), (Ie), (If), (Ig) and (Ih) is hydrogen, fluoro, hydroxyl, or (C₁-C₄)alkanoylamino, provided that when T is N or T is CR³ and R³ is hydroxyl or fluoro, R² is not (C₁-C₃)alkoxy(C₁-C₅)alkoxy, (C₃-C₄)cycloalkyl(C₁-C₅)alkoxy, (C₁-C₃)alkoxy-carbonylamino(C₁-C₅)alkoxy, (C₁-C₃)alkanoylamino(C₁-C₅)alkoxy or (C₁-C₃)alkylaminocarbonyl(C₁-C₅)alkoxy.

Values and specific values for the remainder of the variables are as described above in the 1^{st} specific embodiment.

In a 5^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

G is —NHR⁹ or (C₁-C₃)alkyl-NHR⁹, wherein R⁹ is H or (C₁-C₆)alkyl; and values and specific values for the remainder of the variables are as described above in the 4^{th} specific embodiment.

In a 6^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R² for Structural Formulas (Ia), (Ia′), (Ib), (Ic), (Id), (Ie), (If), (Ig) and (Ih) is (C₁-C₃)alkoxy(C₁-C₅)alkyl, (C₁-C₃)alkoxy(C₁-C₅)alkoxy, (C₁-C₃)alkoxycarbonylamino(C₁-C₅)alkyl, (C₁-C₃)-alkoxycarbonylamino(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, or (C₁-C₅)alkylamino-carboxy(C₁-C₅)alkoxy;

R² for Structural Formulas (Ii), (Ij), (Ik) or (Il) is (C₁-C₃)alkoxy(C₁-C₅)allyl, (C₁-C₃)alkoxycarbonylamino(C₁-C₅)alkyl, aminocarboxy(C₁-C₅)alkyl, or (C₁-C₅)alkylaminocarboxy(C₁-C₅)alkyl; and

values and specific values for the remainder of the variables are as described above in the 5^{th} specific embodiment.

In a 7^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R¹ is phenyl optionally substituted with up to 3 substituents independently selected from the group consisting of fluorine, chlorine, cyano, (C₁-C₃)allyl, halo(C₁-C₃)alkyl, (C₁-C₃)alkoxy, and H₂NCO—;

R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—; and values and specific values for the remainder of the variables are as described above in the 6^{th} specific embodiment.

In a 8^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ik) or (Il) or an enantiomer, a diastereomer or a pharmaceutically acceptable salt thereof, wherein:

R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl; and

R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole;

R² for Structural Formulas (Ia), (Ia′), (Ib), (Ic), (Id), (Ie), (If), (Ig) and (Ih) is 4-methoxybutyl, 4-ethoxybutyl, 4-methoxypentyl, 3-methoxypropoxy, 3-(methoxycarbonylamino)propyl, or 2-(methoxycarbonylamino)ethoxy; and

R² for Structural Formulas (Ii), (Ij), (Ik) and (Il) is R² is 4-methoxybutyl, 4-ethoxybutyl, 4-methoxypentyl, or 3-(methoxycarbonylamino)propyl; and

values and specific values for the remainder of the variables are as described above in the 6^{th} specific embodiment.

In a 9^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ik) or (Il) or an enantiomer, a diastereomer or a pharmaceutically acceptable salt thereof, wherein R³ for Structural Formula (Ia), (Ia′), (Ib), (Ic), (Id), (Ie), (If), (Ig) and (Ih) is hydrogen, hydroxyl or methoxycarbonylamino, provided that when R³ is hydroxyl, R² is not 3-methoxypropoxy, 2-(acetylamino)ethoxy, or 2-(methoxycarbonylamino)ethoxy; values and specific values for the remainder of the variables are as described above in the 8^{th} specific embodiment.

In a 10^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl

R⁷ is methyl, ethyl, propyl, or isopropyl;

R^{a} and R^{b}, for each occurrence, are independently H, methyl or R^{a} and R^{b} attached to one carbon atom taken together are an oxo; and

G is —NHR⁹ or CH₂NHR⁹, wherein R⁹ is H, methyl or ethyl; and

values and specific values for the remainder of the variables are as described above in the 9^{th} specific embodiment.

More specifically, R is phenyl, 3-methylphenyl or 3-ethylphenyl;

R¹ is phenyl or 3-chlorophenyl;

R² is 4-methoxybutyl;

R³ is hydroxyl;

R⁷ is methyl; and

G is —NH₂ or —CH₂NH₂.

In a 11^{th} specific embodiment, the aspartic protease inhibitor of the present invention is represented by the following Structural Formula (Ia), (Ia′), (Ib), (Ic), (Id), (Ie), (If), (Ig), (Ih), (Ii), OD, (Ik) or (Il) or an enantiomer, a diastereomer or a pharmaceutically acceptable salt thereof, wherein:

R is a) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, or halo(C₁-C₃)alkyl; or

b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio or H₂NCO; or

### c) —(CH₂)₄— or —(CH₂)₅—;

R¹ is phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—;

R² for Structural Formulas (Ia), (Ia′), (Ib), (Ic), (Id), (Ie), (If), (Ig) and (Ih) is 3-(acetylamino)propyl or 2-(acetylamino)ethoxy;

R² for Structural Formulas (Ii), (Ij), (Ik) and (Il) is 3-(acetylamino)propyl; and

R³ for Structural Formulas (Ia), (Ia′), (Ib), (Ic), (Id), (Ie), (If), (Ig) and (Ih) is hydrogen, fluoro, hydroxyl, or (C₁-C₄)alkanoylamino, provided that when R³ is hydroxyl or fluoro, R² is not 2-(acetylamino)ethoxy.

Values and specific values for the remainder of the variables are as described above in the 1^{st} specific embodiment.

In a 12^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein G is —NHR⁹ or (C₁-C₃)alkyl-NHR⁹, wherein R⁹ is H or (C₁-C₆)alkyl; and values and specific values for the remainder of the variables are as described above in the 11^{th} specific embodiment.

In a 13^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R¹ is phenyl optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—; and

R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—; and

values and specific values for the remainder of the variables are as described above in the 12^{th} specific embodiment.

In a 14^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl; and

R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole; and

values and specific values for the remainder of the variables are as described above in the 13^{th} specific embodiment.

In 15^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein R³ for Structural Formulas (Ia), (Ia′), (Ib), (Ic), (Id), (Ie), (If), (Ig) and (Ih) is hydrogen, hydroxyl or methoxycarbonylamino, provided that when R³ is hydroxyl, R² is not 2-(acetylamino)ethoxy; and values and specific values for the remainder of the variables are as described above in the 14^{th} specific embodiment.

In a 16^{th} specific embodiment, the aspartic protease inhibitor of the present invention is represented by the following Structural Formula (Ia), (Ia′), (Ib), (Ic), (Id), (Ie), (If), (Ig), (Ih), (Ii), (Ij), (Ik) or (Il) or an enantiomer, a diastereomer or a pharmaceutically acceptable salt thereof, wherein:

R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl;

R⁷ is methyl, ethyl, propyl, or isopropyl;

R^{a} and R^{b}, for each occurrence, are independently H, methyl or R^{a} and R^{b} attached to one carbon atom taken together are an oxo;

G is —NHR⁹ or CH₂NHR⁹, wherein R⁹ is H, methyl or ethyl; and

values and specific values for the remainder of the variables are as described above in the 15^{th} specific embodiment.

More specifically, R is phenyl, 3-methylphenyl or 3-ethylphenyl;

R¹ is phenyl or 3-chlorophenyl;

R² is 4-methoxybutyl;

R³ is hydroxyl;

R⁷ is methyl; and

G is —NH₂ or —CH₂NH₂.

In one embodiment, the aspartic protease inhibitor of the present invention is each of the following compounds or their enantiomers, diastereomers, or pharmaceutically acceptable salts:

| Compd | | |
|---|---|---|
| No. | Structure | Name |
| 1 | 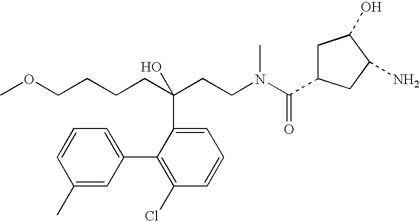 | (1S,3R,4S)-3-amino-N-[3-(6- chloro-3′-methyl-2- biphenylyl)-3-hydroxy-7- (methyloxy)heptyl]-4-hydroxy- N- methylcyclopentanecarboxamide |
| 2 | 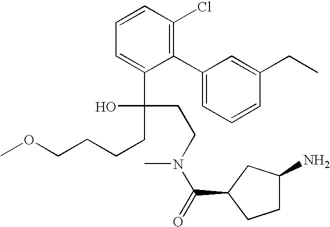 | (1R,3S)-3-amino-N-[3-(6- chloro-3′-ethyl-2-biphenylyl)- 3-hydroxy-7- (methyloxy)heptyl]-N- methylcyclopentanecarboxamide |
| 3 | 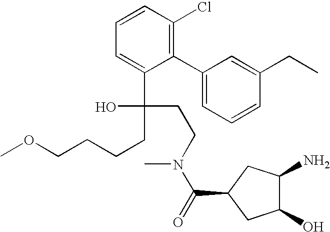 | (1S,3R,4S)-3-amino-N-[3-(6- chloro-3′-ethyl-2-biphenylyl)- 3-hydroxy-7- (methyloxy)heptyl]-4-hydroxy- N- methylcyclopentanecarboxamide |
| 4 | 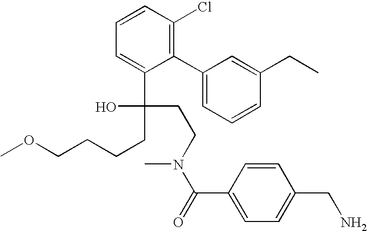 | 4-(aminomethyl)-N-[3-(6- chloro-3 ′-ethyl-2-biphenylyl)- 3-hydroxy-7- (methyloxy)heptyl]-N- methylbenzamide |
| 5 | 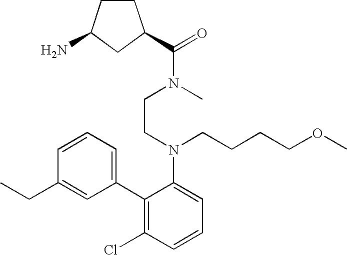 | (1R,3S)-3-amino-N-(2-{(6- chloro-3′-ethyl-2- biphenylyl)[4- (methyloxy)butyl]amino}ethyl)- N- methylcyclopentanecarboxamide |
| 6 | 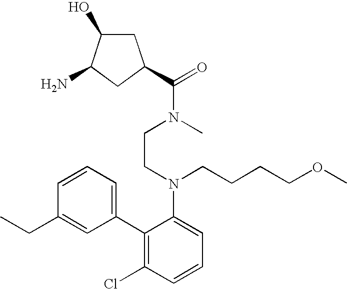 | (1S,3R,4S)-3-amino-N-(2-{(6- chloro-3′-ethyl-2- biphenylyl)[4- (methyloxy)butyl]amino}ethyl)- 4-hydroxy-N- methylcyclopentanecarboxamide |
| 7 | 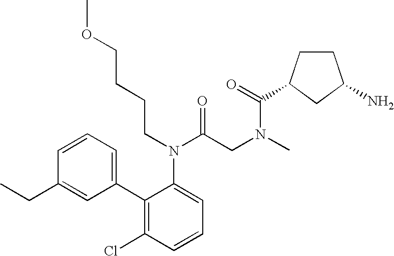 | (1R,3S)-3-amino-N-(2-{(6- chloro-3′-ethyl-2- biphenylyl)[4- (methyloxy)butyl]amino}-2- oxoethyl)-N- methylcyclopentanecarboxamide |
| 8 | 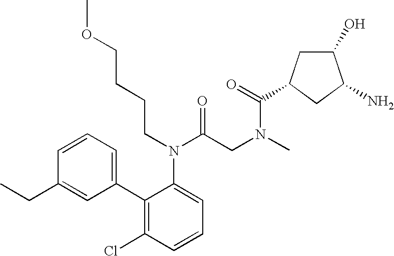 | (1S,3R,48)-3-amino-N-(2-{(6- chloro-3′-ethyl-2- biphenylyl)[4- (methyloxy)butyl]amino}-2- oxoethyl)-4-hydroxy-N- methylcyclopentanecarboxamide |
| 9 | 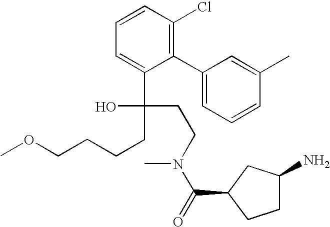 | (1R,3S)-3-amino-N-(3-(6- chloro-3′-methylbiphenyl-2- yl)-3-hydroxy-7- methoxyheptyl)-N- methylcyclopentanecarboxamide |
| 10 | 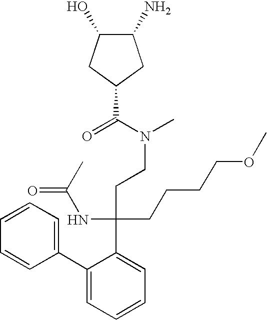 | (1S,3R,4S)-N-(3-acetamido-3- (biphenyl-2-yl)-7- methoxyheptyl)-3-amino-4- hydroxy-N- methylcyclopentanecarboxaimde |
| 11 | 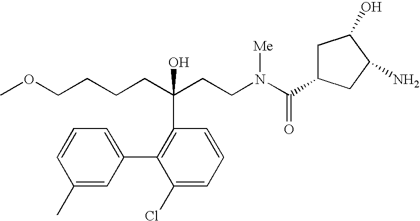 | (1S,3R,4S)-3-amino-N-((S)-3- (6-chloro-5′-methylbiphenyl-2- yl)-3-hydroxy-7- methoxyheptyl)-4-hydroxy-N- methylcyclopentanecarboxamide |

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Alkylene” means —[CH₂]ₓ—, wherein x is a positive integer. x is typically a positive integer from 1-10, more typically from 1-5, even more typically 2-4 and more typically yet from 2-3. Alkylene groups are optionally substituted at any one or more substitutable carbon atom, i.e., a carbon atom that is bonded to a hydrogen, wherein the hydrogen is replaced with a substituent.

“Alkenylene” is an alkylene group in which at least one single bond connecting adjacent methylene groups has been replaced with a double bond. Alkenylene groups are optionally substituted at any one or more substitutable carbon atom, i.e., a carbon atom that is bonded to a hydrogen, wherein the hydrogen is replaced with a substituent.

“Alkynylene” is an alkylene group in which at least one single bond connecting adjacent methylene groups has been replaced with a double bond. Alkynylene groups are optionally substituted at any one or more substitutable carbon atom, i.e., a carbon atom that is bonded to a hydrogen, wherein the hydrogen is replaced with a substituent.

“Cycloalkyl” means a saturated aliphatic cyclic hydrocarbon radical having the specified number of carbon atoms. Thus, (C₃-C₇)cycloalkyl means a radical having from 3-7 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic or polycyclic ring radical containing 1 to 4 heteroatoms independently selected from N, O, and S. Heteroaryl rings include furyl, thienyl, thiophenyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridinyl-N-oxide, pyridazinyl, pyrimidinyl, pyrazinyl, indolizinyl, indolyl, isoindolyl, benzo[b]furyl, benzo[b]thienyl, indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, 1,2,3-triazolyl, 1,2,4-triazolyl, 1,3,4-oxadiazolyl, 1,2,5-thiadiazolyl, 1,2,5-thiadiazolyl-1-oxide, 1,2,5-thiadiazolyl-1,1-dioxide, 1,3,4-thiadiazolyl, 1,2,4-triazinyl, 1,3,5-triazinyl, tetrazolyl, and pteridinyl.

Bicyclic heteroaryl rings are bicyclo[4.4.0] and bicyclo[4.3.0] fused ring systems of which at least one ring is aromatic containing 1 to 4 heteroatoms independently selected from N, O, and S, and include indolizine, indole, isoindole, benzo[b]thiophene, quinoline, isoquinoline, quinazoline, purine, benzothiophene, benzofuran, 2,3-dihydrobenzofuran, cinnoline, phthalazine, benzodioxole, benzimidazole, indazole, benzisoxazole, benzoxazole, and benzothiazole, quinoxaline, 1,8-naphthyridine, and pteridine.

Bicycloalkyl rings are fused, bridged and spiro ring systems and include bicyclo[1.1.0]butane, bicyclo[1.2.0]pentane, bicyclo[2.2.0]hexane, bicyclo[3.2.0]heptane, bicyclo[3.3.0]octane, bicyclo[4.2.0]octane, bicyclo[2.2.1]heptane, bicyclo[2.2.2]octane, bicyclo[3.2.1]octane, bicyclo[3.2.2]nonane, bicyclo[3.3.1]nonane, bicyclo[3.3.2]decane and bicyclo[3.3.3]undecane, spiro[2.2]pentane, spiro[2.3]hexane, spiro[3.3]heptane, spiro[2.4]heptane, spiro[3.4]octane and spiro[2.5]octane.

“Alkoxy” means an alkyl radical attached through an oxygen linking atom. “(C₁-C₄)-alkoxy” includes the methoxy, ethoxy, propoxy, and butoxy.

“Aromatic” means an unsaturated cycloalkyl ring system.

“Aryl” means an aromatic monocyclic or polycyclic ring system. Aryl systems include phenyl, naphthalenyl, fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Oxo” refers to ═O. When an oxo group is a substituent on a carbon atom, they form a carbonyl group (—C(O)—). When one oxo group is a substituent on a sulfur atom, they form a sulfinyl (sulfoxide —S(O)—) group. When two oxo groups are a substituent on a sulfur atom, they form a sulfonyl (sulfone —S(O)₂—) group.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

(i) Enantiomers, Diastereomers, and Salts

Certain compounds of Formula I may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The anionic salt form of a compound of the invention includes the acetate, bromide, camsylate, chloride, edisylate, fumarate, hydrobromide, hydrochloride, iodide, isethionate, lactate, mesylate, maleate, napsylate, salicylate, sulfate, and tosylate salts.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates.” Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by [symbol] which represents [symbol] or

[symbol]

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; atrial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodeling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I, comprise a pharmaceutically acceptable salt of a compound of Formula I or a prodrug or pharmaceutically active metabolite of such a compound or salt and one or more pharmaceutically acceptable carriers therefor.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of Formula I or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of Formula I or composition thereof in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering β-secretase inhibiting compound of Formula I or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of Formula I or composition thereof in a combination therapy with one or more additional agents for the treatment of malaria including artemisinin, chloroquine, halofantrine, hydroxychloroquine, mefloquine, primaquine, pyrimethamine, quinine, sulfadoxine

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally; or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or film-coated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer, which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

### Methods of Preparation

In the discussion below, R, R¹, R², T, R³, A, Q, E, G, W, R⁴, R^{4a}, R⁷, R⁸, R^{a} and R^{b} are defined as described above for compounds of Structural Formula I. In cases where the synthetic intermediates and final products of Structural Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999)

In a first process of the invention, a compound of Structural Formula I, in which a nitrogen atom that is part of A is attached to Q, is prepared by reaction of an amine of Structural Formula II with an intermediate of Structural Formula III:

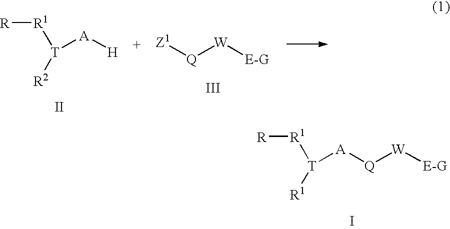

wherein Z¹ in III is a leaving group such as halide, alkanesulfonate, haloalkanesulfonate, arylsulfonate, aryloxide, heteroaryloxide, azole, azolium salt, or alkoxide.

Intermediates of formula II wherein H is attached to a nitrogen atom that is part of A are prepared from intermediates of Formula IV:

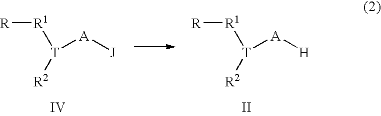

wherein J is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IV wherein T is CR³ and R³═OH are prepared from ketone intermediates of formula V by addition of an organometallic reagent of formula VI, where M is for example Li, MgCl, MgBr, or MgI, to the carbonyl group of V:

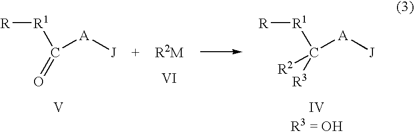

Intermediates of Formula IV wherein R³═H and R² is a group attached by an ether linkage are prepared from alcohol intermediates of formula VII by reaction with an alkylating agent under basic conditions or by reaction with an alcohol of formula R²OH under acidic conditions.

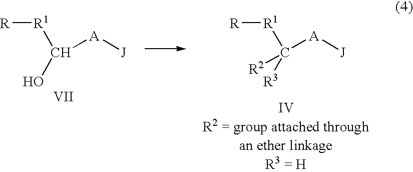

Alcohol intermediates of formula VII are prepared by reduction of ketone intermediates of formula V:

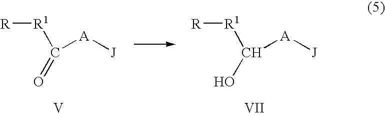

or by addition of an organometallic reagent of formula VIII, wherein M is, for example Li, MgCl, MgBr, or MgI, to an aldehyde of Formula IX:

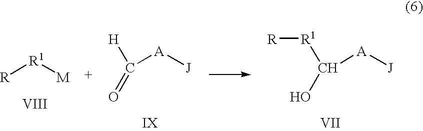

Ketone intermediates of formula V are prepared by the addition of an organometallic reagent of formula VIII, wherein M is Li, MgCl, MgBr, MgI, to a carboxylic acid derivative of formula X wherein Z² is an alkoxy, dialkylamino group, or an N-alkoxy-N-alkylamino group:

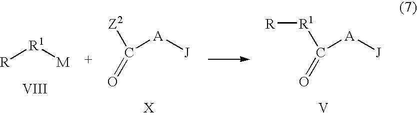

Intermediates of Formula V are also prepared from cuprate organometallic reagents of Formula XI wherein M is Li, MgCl, MgBr or MgI, and a carboxylic acid derivative of Formula X wherein Z² is an alkylthio, arylthio or heteroarylthio group:

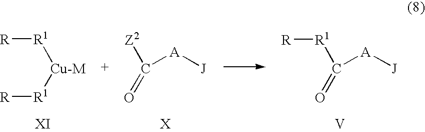

Intermediates of formula V are also prepared by oxidation of alcohol intermediates of formula VII:

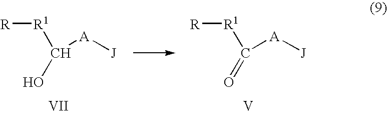

Intermediates of Formula IV, wherein R is an aryl or heteroaryl group, are also prepared by transition metal catalyzed cross coupling of organometallic intermediates of Formula XII, in which M is ZnCl, ZnBr, ZnI, B(OH)₂, pinocolatoboron, or Sn(n-Bu)₃, and intermediates of formula XIII, in which Z³ is a halide or trifluoromethanesulfonate:

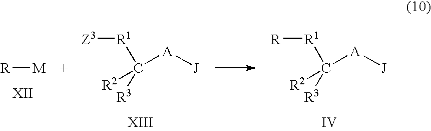

Intermediates of Formula IV, wherein the R is group attached to R¹ through an ether linkage, are also prepared by alkylation of intermediates of formula XIII, in which Z³ is a hydroxyl group with alkylating agents of formula XIV, wherein X is a halogen, alkanesulfonate, haloalkanesulfonate, or arenesulfonate leaving group:

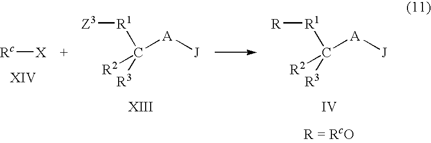

The intermediates of Formula XIII used in reaction schemes 10 and 11 are available by processes analogous to those described for IV (reaction schemes 3 and 4).

Intermediates of Formula IV wherein R² is attached to the molecule through a carbon atom and R³ is H are prepared from intermediates of Formula IV wherein R³ is OH in one step by deoxygenation, for example with Raney nickel, or in two steps by elimination of water followed by hydrogenation:

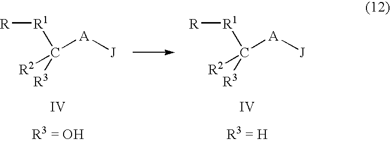

Intermediates of Formula III, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XV:

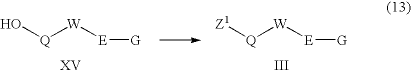

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula III are often prepared and used in situ without isolation.

Intermediates of Formula III, wherein Q is Q1 attached to a nitrogen atom that is part of E and Z¹ is halide, aryloxide, or an azole are prepared by reaction of amine intermediates of Formula XVI with phosgene, aryl chloroformates (e.g., p-nitrophenyl chloroformate or pentafluorophenyl chloroformate), or carbonyl diimidazole respectively. In this reaction, W is a bond.

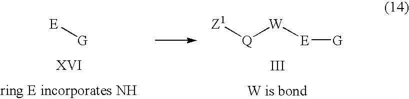

Intermediates of Formula III wherein Q is Q4, Q5, Q6, Q8, Q9 or Q10 attached to a nitrogen atom that is part of E are prepared by reaction of an amine intermediate of Formula XVI with an intermediate of Formula XVII wherein Z¹ is aryloxy, alkoxy, alkylthio, or arylthio. In this reaction, W is a bond.

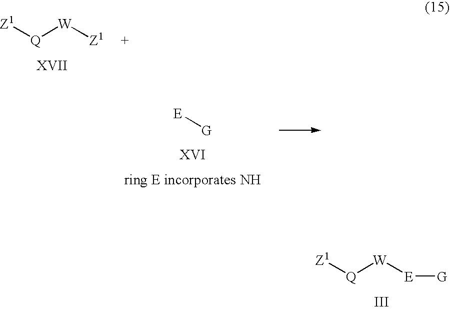

In the second process of the invention, a compound of Formula I, in which a nitrogen atom that is part of E is attached to Q, is prepared by reaction of an intermediate of Formula XVIII and an amine of Formula XVI:

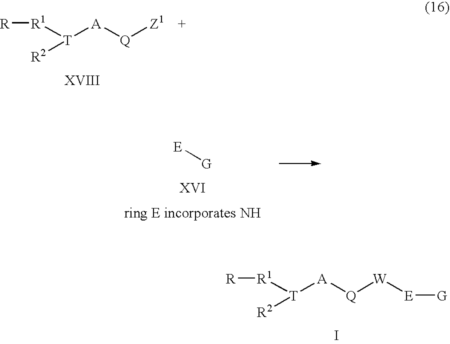

wherein Z¹ is as defined above. In this reaction, W is a bond.

Intermediates of Formula XVIII wherein Q is attached to a nitrogen atom of A and Q is Q1, Q4, Q5, Q6, Q8, Q9, or Q10 are prepared from amine intermediates of Formula II and intermediates of Formula XVII wherein Z¹ is halide, alkanesulfonate, haloalkanesulfonate, carboxylate, arylsulfonate, aryloxy, heteroaryloxy, azole, azolium salt, alkoxy, alkylthio, or arylthio:

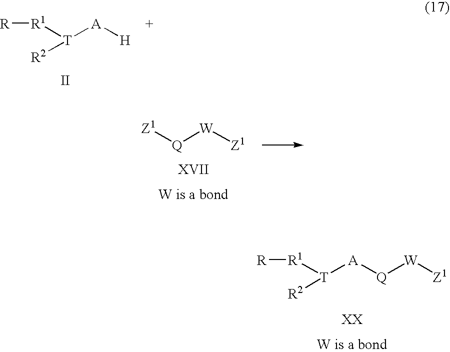

In the third process of the invention, a compound of Formula I in which T is CR³ and R³ is hydroxy is prepared by addition of an organometallic species of Formula VI, wherein M′ is for example Li, MgCl, MgBr, or MgI, to a ketone intermediate of Formula XIX:

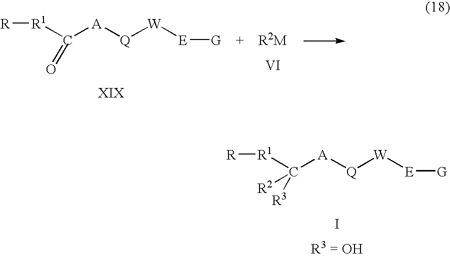

Ketone intermediates of Formula XIX are prepared by processes analogous to those shown for ketone intermediates of formula V in reaction schemes 7, 8, and 9.

In the fourth process of the invention, a compound of Formula I, in which R is an optionally substituted aromatic or heteroaromatic ring, is prepared by transition metal, especially palladium, catalyzed cross coupling of an organometallic species of Formula XXI, wherein M² is for example B(OH)₂, B(OC(Me)₂C(Me₂)O), SnBu₃, or ZnBr, and an intermediate of Formula XXII wherein Z² is Cl, Br, I, or OSO₂CF₃:

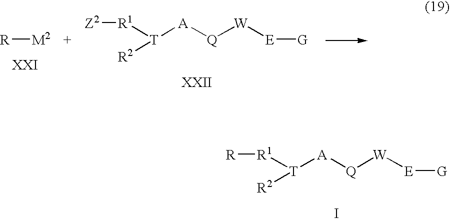

Intermediates of Formula XXII are prepared by processes analogous to those shown for compounds of Formula I in reaction schemes 1, 16, and 18.

In the fifth process of the invention, a compound of Formula I, in which R is an alkoxy, cycloalkoxy, cycloalkylalkoxy or arylalkoxy group, is prepared by reaction of an alkylating agent of Formula XXIII, in which Z³ is chloride, bromide, iodide, methanesulfonate, arenesulfonate or trifluoromethanesulfonate and Rc is an alkyl, cycloalkyl, cycloalkylalkyl or arylalkyl, with a hydroxy compound of Formula XXIV:

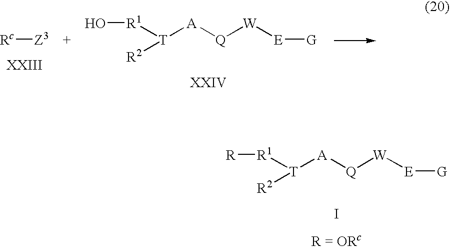

Intermediates of Formula XXIV are prepared by routes analogous to those shown for compounds of Formula I in reaction schemes 1 and 16.

In the sixth process of the invention, a compound of Formula I in which R² is attached through an ether linkage, T is CR³ and R³ is H, A is an aromatic or heteroaromatic ring, and X and Y are single bonds is prepared from an alcohol of Formula XXVI and alcohol of Formula XXV in the presence of acid:

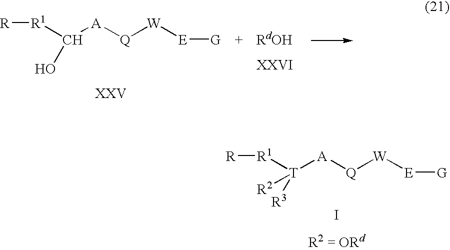

Alcohols of Formula XXV are prepared by reduction of ketones of XIX:

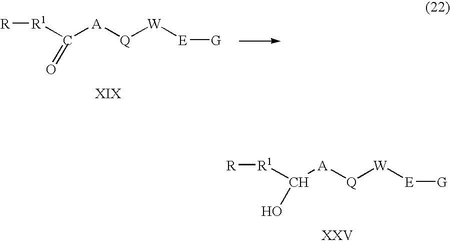

In the seventh process of the invention, a compound of Formula I in which G is an alkylamino group is prepared by reductive alkylation of a compound of Formula I in which G is amino with an aldehyde R^{a}CHO of Formula XXVI wherein R^{a} is alkyl with, for example, NaBH(OAc)₃ or NaBH₃CN:

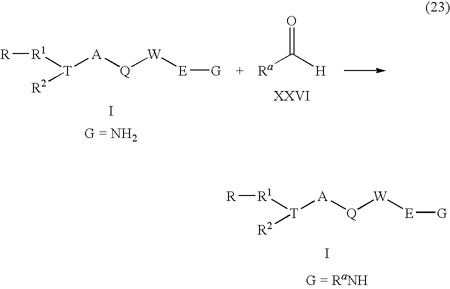

In the eighth process of the invention, a compound of Formula I wherein G is alkylamino is prepared from a compound of Formula I where G is NHMe by reductive alkylation with an aldehyde R^{a}MHO of Formula XXVI wherein R^{a} is alkyl with followed by N-demethylation with a nucleophilic species:

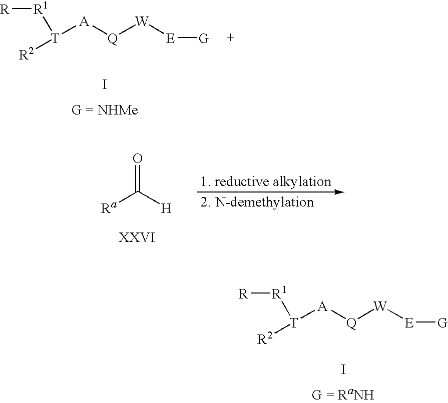

In the ninth process of the invention, a compound of Formula I in which R³═OH is treated with a nitrile XXVII in which R^{a} is alkyl and a strong acid under the conditions of the Ritter reaction to afford a compound of Formula I in which R³═R^{a}CONH:

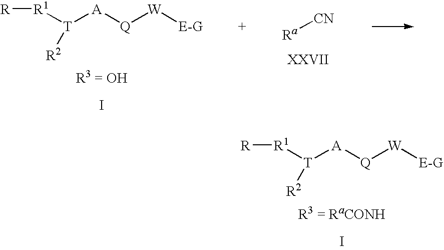

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| Boc | tert-butoxy carbonyl or t-butoxy carbonyl |
| (Boc)₂O | di-tert-butyl dicarbonate |
| brine | saturated aqueous NaCl |
| CH₂Cl₂ | methylene chloride |
| CH₃CN or MeCN | acetonitrile |
| Compd | compound |
| DIEA | N,N-diisopropylethylamine |
| DMAP | 4-(dimethylamino)pyridine |
| DMF | N,N-dimethylformamide |
| EDC | 1-[3-(dimethylamino)propyl]-3-ethylcarbodiimide |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or LiAlH₄ | lithium aluminum hydride |
| LC-MS | liquid chromatography-mass spectroscopy |
| LHMDS | lithium hexamethyldisilazane |
| Me | methyl |
| MeCN | acetonitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin-2,5- |
| | dione |
| TFA | trifluoroacetic acid |
| THF | tetrahydrofuran |
| TMSCl | chlorotrimethylsilane or trimethylsilyl chloride |
| t_{R} | retention time |

### EXEMPLIFICATION

### Purification Methods

Preparative HPLC refers to reverse phase HPLC on a C-18 column eluted with a water/acetonitrile gradient containing 0.01% TFA run on a Gilson 215 system.

Column chromatography and flash chromatography refer to normal phase chromatography on a silica gel column or cartridge eluted with an hexanes/EtOAc gradient.

### Analytical Methods

LC-MS (3 min)

### LC Conditions:

Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

MS Conditions: Electrospray ionization.

### Preparation A

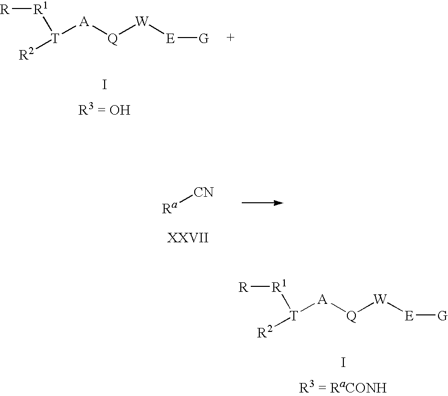

### Step 1. N-{[(1,1-dimethylethyl)oxy]carbonyl}-N-methyl-β-alanine

3-(methylamino)propanenitrile (10 mL, 105.7 mmol) was treated with 200 mL of aqueous 1N NaOH and stirred at 95° C. for 20 h. After the mixture was cooled to room temperature a solution of Boc₂O (34.6 g, 158.5 mmol) in 200 mL of THF was added, and the mixture was stirred at room temperature for 24 h before being washed with Et₂O (2×200 mL) and CH₂Cl₂ (2×200 mL). The aqueous layer was cooled to 0° C., acidified to pH 4 by the addition of aqueous 7N HCl, and extracted with CH₂Cl₂ (2×150 mL). The combined organic extracts were dried (MgSO₄), filtered, and concentrated under reduced pressure to give N-{[(1,1-dimethylethyl)oxy]carbonyl}-N-methyl-β-alanine as a colorless oil (17.53 g, 80%).

### Step 2. N³-{[(1,1-dimethylethyl)oxy]carbonyl}-N¹,N³-dimethyl-N¹-(methyloxy)-β-alaninamide

A solution of N-{[(1,1-dimethylethyl)oxy]carbonyl}-N-methyl-β-alanine (5.0 g, 24.6 mmol), N,O-dimethylhydroxylamine hydrochloride (3.0 g, 30.75 mmol), EDC (5.9 g, 30.75 mmol), and i-PrNEt₂ (10.7 mL, 61.5 mmol) in 100 mL of dichloromethane was stirred at 25° C. overnight before being diluted with 300 mL of EtOAc. The mixture was washed with aqueous 0.5 N HCl and saturated aqueous NaHCO₃, dried (MgSO₄), and concentrated under reduced pressure to provide N³-{[(1,1-dimethylethyl)oxy]carbonyl}-N¹,N³-dimethyl-N¹-(methyloxy)-β-alaninamide as a colorless oil (3.6 g, 59%).

### Step 3. 1,1-dimethylethyl methyl[7-(methyloxy)-3-oxoheptyl]carbamate

A solution of N³-{[(1,1-dimethylethyl)oxy]carbonyl}-N¹-methyl-N¹-(methyloxy)-β-alaninamide (1.0 g, 4.06 mmol) in 5 mL of THF at −15° C. was treated with a solution of 4-(methyloxy)butylmagnesium chloride (10 mL of 1 M in THF, 10 mmol) and the mixture was stirred at room temperature for 90 min before being quenched with the addition of 20 mL of saturated aqueous NH₄Cl and extracted with Et₂O (3×20 mL). The combined organic extracts were dried (MgSO₄), and concentrated to provide 1,1-dimethylethyl methyl[7-(methyloxy)-3-oxoheptyl]carbamate as a colorless oil (1.23 g).

### Step 4. 1,1-dimethylethyl [3-(6-chloro-3′-ethyl-2-biphenylyl)-3-hydroxy-7-(methyloxy)heptyl]methylcarbamate

A solution of 2-bromo-6-chloro-3′-ethylbiphenyl (0.48 g, 1.6 mmol) in 4 mL of THF at −78° C. was treated with t-BuLi (1.9 mL of 1.7 M in pentane, 3.2 mmol) and stirred for 20 min before 1,1-dimethylethyl methyl[7-(methyloxy)-3-oxoheptyl]carbamate (0.22 g, 0.8 mmol) was added. The mixture was stirred at −78° C. for 30 min then warmed to 0° C. before being quenched with the addition of 15 mL of saturated aqueous NH₄Cl solution and extracted with Et₂O (3×20 mL). The combined organic extracts were dried (MgSO₄) and concentrated to provide 1,1-dimethylethyl [3-(6-chloro-3′-ethyl-2-biphenylyl)-3-hydroxy-7-(methyloxy)heptyl]methylcarbamate as a colorless oil (0.63 g).

### Step 5. 3-(6-chloro-3′-ethyl-2-biphenylyl)-1-(methylamino)-7-(methyloxy)-3-heptanol

1,1-dimethylethyl [3-(6-chloro-3′-ethyl-2-biphenylyl)-3-hydroxy-7-(methyloxy)heptyl]methylcarbamate was treated with 8 mL of 1:1 mixture of aqueous 2N HCl and acetronitrile and stirred overnight before being concentrated under reduced pressure and purified on an SCX resin cartridge to provide 3-(6-chloro-3′-ethyl-2-biphenylyl)-1-(methylamino)-7-(methyloxy)-3-heptanol as a viscous oil (0.19 g).

The following compounds were prepared following procedures analogous to those described above:
1) 3-(6-chloro-3′-methyl-2-biphenylyl)-1-(methylamino)-7-(methyloxy)-3-heptanol

### Preparation B

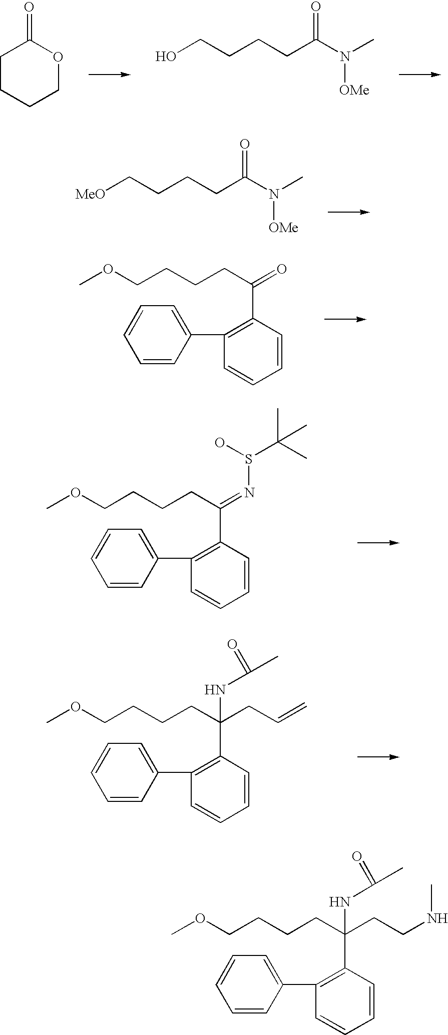

### Step 1. 5-hydroxy-N-methoxy-N-methylpentanamide

To a stirred suspension of N,O-dimethylhydroxylamine hydrochloride (14.6 g, 150 mmol) in dry dichloromethane (150 mL) was added trimethylaluminum (2.0 M in toluene, 75 mL, 150 mmol) drop wise at room temperature. After stirring for 30 mins, tetrahydropyran-2-one (10 g, 100 mmol) in dichloromethane (50 mL) was added drop wise. The reaction mixture was stirred over night at room temperature. At 0° C., 1 N HCl (80 mL) was added slowly (exothermic reaction) then the reaction mixture was extracted with dichloromethane (3×200 mL). The combined organic phases were dried over sodium sulfate, filtered and concentrated. The residue was purified by column chromatography on silica gel (120 g column, dichloromethane to 20% methanol in dichloromethane) to give product (9.7 g, 60%).

¹H NMR (CDCl₃) δ 3.68 (s, 3H), 3.63 (t, J=6.1 Hz, 2H), 3.17 (s, 3H), 2.46 (t, J=7.0 Hz, 2H), 1.69-1.74 (m, 2H), 1.58-1.64 (m, 2H); MS EI m/z 162.21 (M+H)⁺.

### Step 2. N,5-dimethoxy-N-methylpentanamide

To a solution of 5-hydroxy-N-methoxy-N-methylpentanamide (1.0 g, 6.2 mmol) in DMF (10 mL) at 0° C. was added sodium hydride (60% in mineral oil, 0.32 g, 8.0 mmol). After 30 min, iodomethane (0.5 mL, 8.0 mmol) was added and the reaction mixture was stirred at room temperature overnight. The solvent was removed under reduced pressure. The residue was partitioned between dichloromethane and brine, separated. The aqueous phase was extracted with dichloromethane twice. The combined organic phases were dried over sodium sulfate, filtered and concentrated. The residue was purified by column chromatography on silica gel (40 g column, ethyl acetate) to give product (0.7 g, 64%). ¹H NMR (CDCl₃) δ 3.68 (s, 3H), 3.39 (t, J=6.4 Hz, 2H), 3.32 (s, 3H), 3.17 (s, 3H), 2.45 (t, J=6.8 Hz, 2H), 1.58-1.74 (m, 4H); MS EI m/z 176.23 (M+H)⁺.

### Step 3. 1-(biphenyl-2-yl)-5-methoxypentan-1-one

A stirred solution of 2-bromobiphenyl (1.1 g, 4.6 mmol) in dry THF (10 mL) was cooled to −78° C. and a solution of tert-BuLi (1.7 M in pentane, 5.5 mL, 9.3 mmol) was added drop wise. After 30 min, a solution of N,5-dimethoxy-N-methylpentanamide

(0.54 g, 3.1 mmol) in dry THF (3 mL) was added. The cooling bath was allowed to warm to −20° C. and the reaction mixture was stored in −20° C. freezer overnight. After warming to room temperature, 1 N HCl (50 mL) was added to the reaction mixture. The mixture was extracted with ethyl acetate (2×100 mL). The combined organic extracts were washed with brine, dried over Na₂SO₄, filtered and concentrated. The residue was purified by column chromatography on silica gel (40 g column, 0% ethyl acetate in hexanes to 30% ethyl acetate in hexanes) to give product (0.24 g, 29%). ¹H NMR (CDCl₃) δ 7.31-7.52 (m, 9H), 3.24 (s, 3H), 3.18 (t, J=6.4 Hz, 2H), 2.25 (t, J=7.6 Hz, 2H), 1.44-1.53 (m, 2H), 1.30-1.37 (m, 2H); MS EI m/z 291.19 (M+Na)⁺.

### Step 4. N-[1-(biphenyl-2-yl)-5-methoxy-pentylidene]-tert-butanesulfinamide

To a mixture of 1-(biphenyl-2-yl)-5-methoxypentan-1-one (240 mg, 0.89 mmol) and titanium ethoxide (0.55 mL, 2.67 mmol) in dry 1,4-dioxane (1 mL) in seal tube under nitrogen was added 2-methyl-2-propanesulfinamide (216 mg, 1.78 mmol). The tube was sealed and heated at 90° C. for 72 h. After cooling to room temperature, the reaction mixture was poured into a stirred mixture of ethyl acetate and brine, stirred for 2 h, filtered through Celite, separated. The aqueous phase was extracted with ethyl acetate twice. The combined organic phases were dried over sodium sulfate, filtered and concentrated. The residue was purified by column chromatography on silica gel (40 g column, 0% ethyl acetate in hexanes to 50% ethyl acetate in hexanes) to give product (57 mg, 17%) ¹H NMR (CDCl₃) δ 7.31-7.8 (m, 9H), 3.14-3.36 (m, 5H), 1.80-2.40 (m, 2H), 1.20-1.60 (m, 13H); MS EI m/z 372.30 (M+H)⁺.

### Step 5. N-(4-(biphenyl-2-yl)-8-methoxyoct-1-en-4-yl)acetamide

To a solution of N-[1-(biphenyl-2-yl)-5-methoxy-pentylidene]-tert-butanesulfinamide (57 mg, 0.15 mmol) in dry THF (1 mL) at room temperature was added allylmagnesium bromide (1.0 M, 0.45 mL, 0.45 mmol) and the reaction mixture was stirred over night. The reaction mixture was poured into a mixture of ethyl acetate and brine, separated. The organic phase was dried over sodium sulfate, filtered and concentrated. The residue was dissolved in methanol (2 mL), added 1N HCl (0.5 mL) and stirred overnight. The reaction mixture was then concentrated, co-evaporated with acetonitrile three times. The residue was dissolved in dichloromethane (2 mL), added acetyl chloride (0.05 mL, 0.75 mmol) and triethylamine (0.2 mL, 1.5 mmol). After 1 h, the reaction mixture was poured into a mixture of dichloromethane and brine, separated. The organic phase was dried over sodium sulfate, filtered and concentrated. The residue was purified by preparative HPLC (C-18 column, 2 to 95% CH₃CN in H₂O containing 0.01% CF₃CO₂H over 20 min, 20 mL min⁻¹) to afford product (20 mg, 32%). ¹H NMR (CD₃OD) δ 7.25-7.50 (m, 6H), 7.14-7.22 (m, 2H), 6.92 (d, J=7.3 Hz, 1H), 5.52-5.64 (m, 1H), 4.97-5.04 (m, 2H), 3.29-3.34 (m, 2H), 3.27 (s, 3H), 3.03 (dd, J=13.4, 7.3 Hz, 1H), 2.66 (dd, J=13.1, 7.0 Hz, 1H), 1.92-2.02 (m, 1H), 1.76-1.84 (m, 1H), 1.60 (s, 3H), 1.44-1.50 (m, 2H), 1.02-1.24 (m, 2H); MS EI Ink 352.31 (M+H)⁺.

### Step 6. N-(3-(biphenyl-2-yl)-7-methoxy-1-(methylamino)heptan-3-yl)acetamide

To a solution of N-(4-(biphenyl-2-yl)-8-methoxyoct-1-en-4-yl)acetamide (20 mg, 0.057 mmol) in THF/H₂O (3 mL/1 mL) was added sodium periodate (36.6 mg, 0.17 mmol) followed by a solution of osmium tetroxide (2.5 wt. % in 2-propanol, 0.03 mL, 0.003 mmol). The reaction mixture was stirred over night and then diluted with ethyl acetate, washed with water then brine. The organic phase was dried over sodium sulfate, filtered and concentrated. The residue was dissolved in methanol (3 mL), added methylamine (33% solution in ethanol, 0.06 mL, 0.57 mmol), methylamine hydrogen chloride (7.7 mg, 0.12 mmol) and sodium cyanoborohydride (10.6 mg, 0.18 mmol). The reaction mixture was stirred over night then purified by reverse phase HPLC to give product as TFA salt (11 mg, 52%). NMR (CD₃OD) δ 7.16-7.48 (m, 8H), 6.96-7.00 (m, 1H), 3.29-3.34 (m, 2H), 3.27 (s, 3H), 2.52-3.18 (m, 6H), 2.24-2.34 (m, 1H), 1.44-1.98 (m, 7H), 1.02-1.24 (m, 2H); MS EI m/z 369.50 (M+H)⁺.

### Preparation C

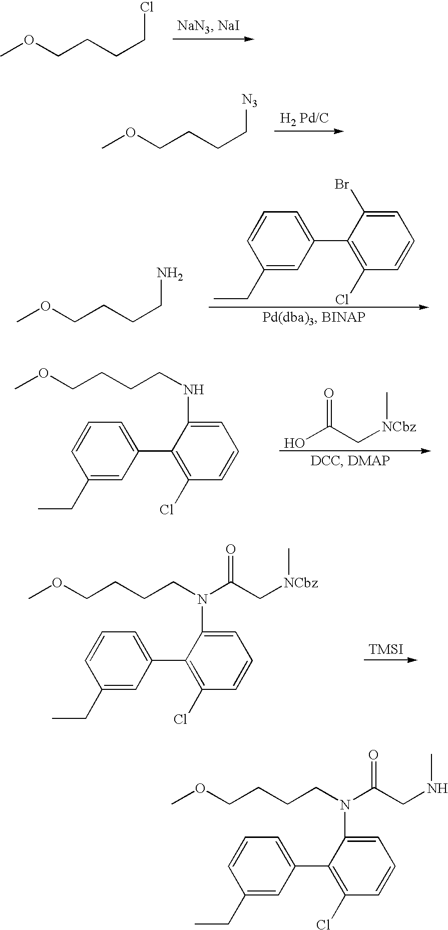

### Step 1. 4-azidobutyl methyl ether

A solution of 1-chloro-4-(methyloxy)butane (10.0 g, 81.6 mmol) in 50 mL of DMSO was treated with sodium azide (16.0 g, 244.8 mmol) and sodium iodide (1.2 g, 8.2 mmol) and stirred at 55° C. for 4 h before being quenched with the addition of 150 mL of water and extracted with EtOAc (3×150 mL). The combined organic layers were washed with 100 mL of brine, dried (MgSO₄), and concentrated under reduced pressure to provide 4-azidobutyl methyl ether as a colorless oil (7.10 g, 68%).

### Step 2. 4-(methyloxy)-1-butanamine

A solution of 1-azido-4-(methyloxy)butane (7.1 g, 55.0 mmol) in 50 mL of ethanol was treated with 3.0 g of 5% Pd/C and the mixture was placed under an atmosphere of hydrogen (45 psi) for 20 h. The mixture was filtered through Celite and concentrated under reduced pressure to give a 4-(methyloxy)-1-butanamine as a colorless liquid.

### Step 3. (6-chloro-3′-ethyl-2-biphenylyl)[4-(methyloxy)butyl]amine

A solution of 2-bromo-6-chloro-3′-ethylbiphenyl (2.61 g, 8.83 mmol) and [4-(methyloxy)butyl]amine (2.73 g, 25.5 mmol) in 20 mL of toluene was treated with Pd(dba)₃ (0.32 g, 0.35 mmol), BINAP (0.33 g, 0.53 mmol), and t-BuOK (4.0 g, 12.4 mmol) and stirred at 100° C. for 20 h. The mixture was filtered through Celite, water was added, and the mixture was extracted with EtOAc (4×50 mL). The combined organic extracts were dried (MgSO₄), concentrated under reduced pressure, and subjected to flash chromatography to provide (6-chloro-3′-ethyl-2-biphenylyl)[4-(methyloxy)butyl]amine as a light brown oil (0.89 g, 32%).

### Step 4. phenylmethyl (2-{(6-chloro-3′-ethyl-2-biphenylyl)[4-(methyloxy)butyl]amino}-2-oxoethyl)methylcarbamate

A solution of Cbz-sarcosine (2.0 g, 8.8 mmol) in 20 mL of CH₂Cl₂ at 0° C. was treated with DCC (1.0 g, 4.8 mmol) and stirred for 30 min. The mixture was filtered and before being added to a solution of (6-chloro-3′-ethyl-2-biphenylyl)[4-(methyloxy)butyl]amine (0.28 g, 0.88 mmol) and DMAP in 4 mL of DMF. The reaction stirred at reflux for 48 h before being quenched with the addition of 50 mL of water and extracted with EtOAc. The organic extract was dried (MgSO₄), concentrated under reduced pressure, and subjected to reverse-phase HPLC to provide phenylmethyl (2-{(6-chloro-3¹-ethyl-2-biphenylyl)[4-(methyloxy)butyl]amino}-2-oxoethyl)methylcarbamate as a colorless oil.

### Step 5. N¹-(6-chloro-3′-ethyl-2-biphenylyl)-N²-methyl-N¹-[4-(methyloxy)butyl]glycinamide

solution of phenylmethyl (2-{(6-chloro-3′-ethyl-2-biphenylyl)[4-(methyloxy)butyl]amino}-2-oxoethyl)methylcarbamate (0.3 g, 0.57 mmol) in 10 mL of acetonitrile at 0° C. was treated with TMSI (0.3 mL, 2.28 mmol) and stirred for 30 min before being concentrated under reduced pressure. The residue was treated with 10 mL of aqueous 2N HCl and washed with Et₂O (2×15 mL). The aqueous layer was cooled to 0° C., basified with the addition of NaOH, and extracted with CH₂Cl₂ (2×20 mL). The combined organic extracts were dried (MgSO₄) and concentrated under reduced pressure to provide N¹-(6-chloro-3¹-ethyl-2-biphenylyl)-N²-methyl-N¹-[4-(methyloxy)butyl]glycinamide as a viscous oil (104 mg, 47%).

### Preparation D

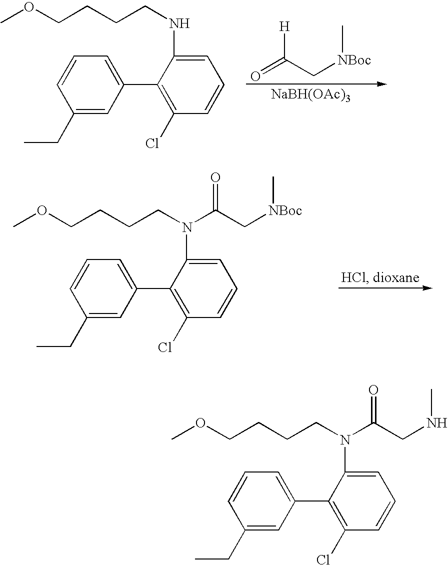

### Step 1. 1,1-dimethylethyl (2-{(6-chloro-3′-ethyl-2-biphenylyl)[4-(methyl oxy)butyl]amino}ethyl)methylcarbamate

A solution of (6-chloro-3′-ethyl-2-biphenylyl)[4-(methyloxy)butyl]amine (0.2 g, 0.63 mmol) and 1,1-dimethylethyl methyl(2-oxoethyl)carbamate (0.33 g, 1.89 mmol) in CHCl₃ at 65° C. was treated with acetic acid (0.04 mL, 0.63 mmol) followed by sodium triacetoxyborohydride (0.53 g, 2.52 mmol) added in portions. The reaction mixture was stirred at 65° C. overnight before being quenched with the addition of 6 mL of aqueous 2N NaOH and extracted with CH₂Cl₂ (2×15 mL). The combined organic extracts were dried (MgSO₄), concentrated under reduced pressure, and subjected to flash chromatography to provide 1,1-dimethylethyl (2-{(6-chloro-3′-ethyl-2-biphenylyl)[4-(methyloxy)butyl]amino}ethyl)methylcarbamate as a colorless oil (0.26 g, 87%).

### Step 2. (6-chloro-3′-ethyl-2-biphenylyl)[2-(methylamino)ethyl][4-(methyloxy)butyl]amine

A solution of 1,1-dimethylethyl (2-{(6-chloro-3′-ethyl-2-biphenylyl)[4-(methyloxy)butyl]amino}ethyl)methylcarbamate (0.26 g, 0.55 mmol) in 3 mL of acetonitrile at 25° C. was treated with 3 mL of 4N HCl in dioxane and stirred for 3 h before being concentrated under reduced pressure. The crude material was purified using an SCX resin cartridge to give (6-chloro-3′-ethyl-2-biphenylyl)[2-(methylamino)ethyl][4-(methyloxy)butyl]amine as a viscous oil (0.2 g, 98%).

### Example 1

### (1S,3R,4S)-3-amino-N-[3-(6-chloro-3′-methyl-2-biphenylyl)-3-hydroxy-7-(methyloxy)heptyl]-4-hydroxy-N-methylcyclopentanecarboxamide (Compd No. 1)

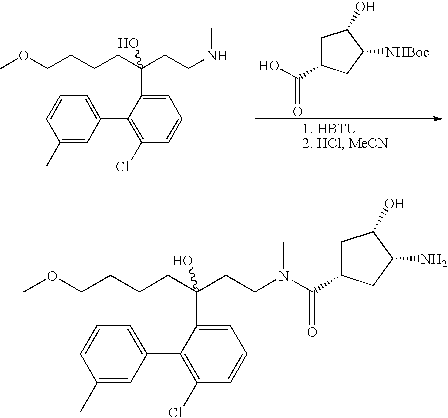

### Step 1. 1,1-dimethylethyl ((1R,2S,45)-4-{[[3-(6-chloro-3′-methyl-2-biphenylyl)-3-hydroxy-7-(methyloxy)heptyl](methyl)amino]carbonyl}-2-hydroxycyclopentyl)carbamate

A solution of 3-(6-chloro-3′-methyl-2-biphenylyl)-1-(methylamino)-7-(methyloxy)-3-heptanol (50 mg, 0.12 mmol) in 0.5 mL of DMF at 25° C. was treated with (1S,3R,4S)-3-({[(1,1-dimethylethyl)oxy]carbonyl}amino)-4-hydroxycyclopentanecarboxylic acid (29 mg, 0.12 mmol), i-Pr₂NEt (0.098 mL, 0.6 mmol), HBTU (45 mg, 0.12 mmol), and HOBt (16 mg, 0.12 mmol) and stirred overnight. The mixture was poured into water and extracted with EtOAc. The combined organic extracts were washed (1N HCl, 1N NaOH, H₂O, brine), dried (Na₂SO₄), concentrated under reduced pressure, and subjected to flash chromatography to provide 1,1-dimethylethyl a 1R,2S,4S)-4-{[[3-(6-chloro-3′-methyl-2-biphenylyl)-3-hydroxy-7-(methyloxy)heptyl](methyl)amino]carbonyl}-2-hydroxycyclopentyl)carbamate as a colorless oil (42 mg, 58%).

### Step 2. (1S,3R,4S)-3-amino-N-[3-(6-chloro-3′-methyl-2-biphenylyl)-3-hydroxy-7-(methyloxy)heptyl]-4-hydroxy-N-methylcyclopentanecarboxamide

A solution of 1,1-dimethylethyl ((1R,2S,4S)-4-{[[3-(6-chloro-3′-methyl-3-biphenylyl)-3-hydroxy-7-(methyloxy)heptyl]methyl)amino]carbonyl}-2-hydroxycyclopentyl)carbamate (20 mg, 0.033 mmol) in 2 mL of CH₃CN at 25° C. was treated with 2 mL of aqueous 2N HCl and stirred overnight. The mixture was concentrated under reduced pressure to provide a colorless oil, which was dissolved in water and lyophilized to provide (1S,3R,4S)-3-amino-N-[3-(6-chloro-3′-methyl-2-biphenylyl)-3-hydroxy-7-(methyloxy)heptyl]-4-hydroxy-N-methylcyclopentanecarboxamide as a white solid (15.7 mg, 88%). MS m/z=503.2 (M⁺).

The following compounds were prepared by procedures analogous to those described in Example 1:

| | |
|---|---|
| Compd No. 2 | (1R,3S)-3-amino-N-[3-(6-chloro-3′-ethyl-2-biphenylyl)-3-hydroxy-7- |
| | (methyloxy)heptyl]-N-methylcyclopentanecarboxamide |
| Compd No. 3 | (1S,3R,4S)-3-amino-N-[3-(6-chloro-3′-ethyl-2-biphenylyl)-3-hydroxy-7- |
| | (methyloxy)heptyl]-4-hydroxy-N-methylcyclopentanecarboxamide |
| Compd No. 4 | 4-(aminomethyl)-N-[3-(6-chloro-3′-ethyl-2-biphenylyl)-3-hydroxy-7- |
| | (methyloxy)heptyl]-N-methylbenzamide |
| Compd No. 5 | (1R,3S)-3-amino-N-(2-{(6-chloro-3′-ethyl-2-biphenylyl)[4- |
| | (methyloxy)butyl]amino}ethyl)-N-methylcyclopentanecarboxamide |
| Compd No. 6 | (1S,3R,4S)-3-amino-N-(2-{(6-chloro-3′-ethyl-2-biphenylyl)[4- |
| | (methyloxy)butyl]amino}ethyl)-4-hydroxy-N- |
| | methylcyclopentanecarboxamide |
| Compd No. 7 | (1R,3S)-3-amino-N-(2-{(6-chloro-3′-ethyl-2-biphenylyl)[4- |
| | (methyloxy)butyl]amino}-2-oxoethyl)-N- |
| | methylcyclopentanecarboxamide |
| Compd No. 8 | (1S,3R,4S)-3-amino-N-(2-{(6-chloro-3′-ethyl-2-biphenylyl)[4- |
| | (methyloxy)butyl]amino}-2-oxoethyl)-4-hydroxy-N- |
| | methylcyclopentanecarboxamide |
| Compd No. 9 | (1R,3S)-3-amino-N-(3-(6-chloro-3′-methylbiphenyl-2-yl)-3-hydroxy-7- |
| | methoxyheptyl)-N-methylcyclopentanecarboxamide |
| Compd No. | (1S,3R,4S)-N-(3-acetamido-3-(biphenyl-2-yl)-7-methoxyheptyl)-3- |
| 10 | amino-4-hydroxy-N-methylcyclopentanecarboxamide |
| | MS m/z = 496.4 (M + H+); ¹H NMR (CD₃OD) δ 7.14-7.48 (m, 8H), |
| | 6.94-7.00 (m, 1H), 4.18-4.30 (m, 1H), 3.40-3.54 (m, 1H), 3.24-3.40 (m, |
| | 5H), 2.84-3.20 (m, 4H), 1.65-2.60 (m, 13H), 1.46-1.54 (m, 2H), |
| | 1.04-1.30 (m, 2H) |

### Example 2

### (1S,3R,4S)-3-amino-N—((S)-3-(6-chloro-3′-methylbiphenyl-2-yl)-3-hydroxy-7-methoxyheptyl)-4-hydroxy-N-methylcyclopentanecarboxamide (Compd No. 11)

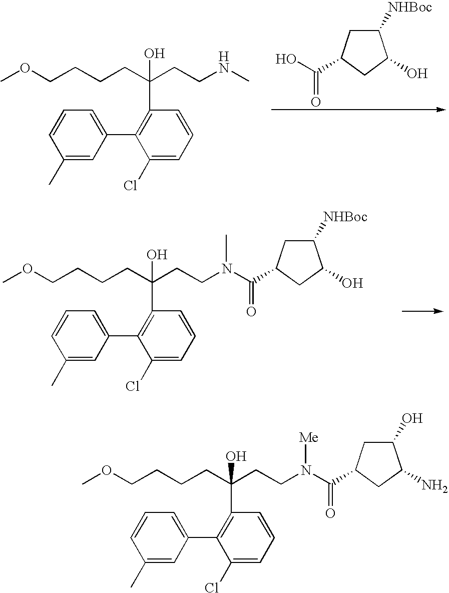

### Step 1. tert-butyl (1S,2R,4R)-4-(3-(6-chloro-5′-methylbiphenyl-2-yl)-3-hydroxy-7-methoxyheptyl)(methyl)carbamoyl)-2-hydroxycyclopentylcarbamate

A solution of 3-(6-chloro-5′-methylbiphenyl-2-yl)-7-methoxy-1-(methylamino)heptan-3-ol (92 mg, 0.245 mmol), (1R,3S,4R)-3-(tert-butoxycarbonylamino)-4-hydroxycyclopentanecarboxylic acid (60 mg, 0.245 mmol), EDCI (117 mg, 0.612 mmol) and HOBt (83 mg, 0.612 mmol) in anhydrous CH₂Cl₂ (8 mL) was treated with DIEA (79 mg, 0.612 mmol) at 0° C. After the addition, the reaction mixture was stirred at room temperature for overnight. The reaction solvent was removed in vacuo. The residue was purified via preparative TLC to give tert-butyl (1S,2R,4R)-4-(3-(6-chloro-5′-methylbiphenyl-2-yl)-3-hydroxy-7-methoxyheptyl)(methyl)carbamoyl)-2-hydroxycyclopentylcarbamate (60 mg, 42%). MS (H⁺): 603.

### Step 2. (1S,3R,48)-3-amino-N—((S)-3-(6-chloro-5′-methylbiphenyl-2-yl)-3-hydroxy-7-methoxyheptyl)-4-hydroxy-N-methylcyclopentanecarboxamide

tert-Butyl(1S,2R,4R)-4-(3-(6-chloro-3¹-methylbiphenyl-2-yl)-3-hydroxy-7-methoxyheptyl)(methyl)carbamoyl)-2-hydroxycyclopentylcarbamate (60 mg) was dissolved in a solution of 20% (V/V) TFA/CH₂Cl₂ (6 mL). The reaction mixture was concentrated in vacuo. The residue was purified by preparative HPLC to afford (1S,3R,4S)-3-amino-N—((S)-3-(6-chloro-5′-methylbiphenyl-2-yl)-3-hydroxy-7-methoxyheptyl)-4-hydroxy-N-methylcyclopentanecarboxamide (2.49 mg, 27%). ¹H NMR (CDCl₃) δ 1.00 (m, 1H), 1.39 (m, 6H), 1.95 (m, 2H), 2.37 (s, 3H), 2.81 (m, 1H), 3.00 (m, 3H), 3.25 (m 3H), 3.50 (m, 2H), 4.25 (m, 1H), 6.98 (m, 1H), 7.18-7.43 (m, 5H), 7.81 (m, 1H).

### Example 3

### In Vitro Activity Studies

### IC₅₀ for Renin

The compounds of the invention have enzyme-inhibiting properties. In particular, they inhibit the action of the natural enzyme renin. The latter passes from the kidneys into the blood where it effects the cleavage of angiotensinogen, releasing the decapeptide angiotensin I, which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors in vitro is demonstrated experimentally by means of a test that measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increases as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

### Example 4

### In Vitro Activity Studies

### IC₅₀ for Renin

All reactions are carried out in a low volume, black, 384 well microtiter plate (Greiner Bio-one). Compounds were diluted in 100% DMSO, and a 100 nL aliquot of each compound concentration was stamped into the plate using a Hummingbird (Genomic Solutions). 5 μL of 600 pM renin (trypsin-activated recombinant human renin) was then added to the plate, followed by 5 μL of 2 μM substrate (Arg-Glu-Lys(5-FAM)-Ile-His-Pro-Phe-His-Leu-Val-Ile-His-Thr-Lys(5,6-TAMRA)-Arg-CONH₂). Both renin and substrate were made up in buffer containing 50 mM HEPES, 125 mM NaCl, 0.1% CHAPS, with the pH adjusted to 7.4. After 2 hours of reaction at room temperature, the plates were read on a Viewlux™ (Perkin Elmer) with an excitation/emission of 485/530 nm, and using a 505 nm cutoff filter. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor.

### Example 5

### In Vitro Activity of the Disclosed Compounds in Human Plasma

The action of renin inhibitors in vitro in human plasma is demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin IS added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are used for deriving percent of inhibition for each concentration of inhibitors and fitting dose-response data into a four parametric model from which IC₅₀ values, defined as concentrations of inhibitors at which 50% inhibition occurs, is determined.

### Example 6

### IC₅₀ Values of the Disclosed Compounds for Renin

The IC₅₀ values of the disclosed compounds for renin were determined according to the protocols described in Example 3 or 4. In these in vitro systems the compounds of the invention exhibit 50% inhibition at concentrations of from approximately 5000 nM to approximately 0.01 nM. Preferred compounds of the invention exhibit 50% inhibition at concentrations of from approximately 50 n M to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma as described in Example 5. According to the IC₅₀ values, compounds 1 and 11 are preferred compounds of the invention.

### Example 7

### Efficacy of the Disclosed Inhibitors in a Transgenic Rat Model

The efficacy of the renin inhibitors may also be evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments could be conducted in 5-10 week-old double transgenic rats (dTGRs). The model has been described in detail earlier. Briefly, the human renin construct that may be used to generate transgenic animals (hRen) is made up of the entire genomic human renin gene (10 exons and 9 introns), with 3.0 kB of the 5′-promoter region and 1.2 kB of 3′ additional sequences. A human angiotensinogen construct containing the entire human angiotensinogen gene (5 exons and 4 introns), with 1.3 kB of 5′-flanking and 2.4 kB of 3′-flanking sequences may be used to generate rats producing human angiotensinogen (hAogen). The hRen and hAogen rats may be rederived using embryo transfer from breeding pairs obtained under license from Ascencion Gmbh (Germany). The hAogen and hRen may then be crossed to produce the double transgenic dTGR) off-spring. The dTGr rats should be maintained on irradiated rodent chow (5VO2, Purina Mills Inc) and normal water. Radio telemetry transmitters (TA11PAC40, Data Sciences International) may be surgically implanted at 5-6 weeks of age. The telemetry system can provide 24-h recordings of systolic, mean, diastolic arterial pressure (SAP, MAP, DAP, respectively) and heart rate (HR). Prior to dosing, baseline hemodynamic measures should be obtained for 24 hours. Rats may then be dosed orally with vehicle or drug and monitored up to 48 hours post-dose.

### Example 8

### In Vivo Activity

The cardiac and systemic hemodynamic efficacy of selective renin inhibitors can be evaluated in vivo in sodium-depleted, normotensive cynomolgus monkeys and in sodium-depleted, normotensive beagle dogs following a single oral and intravenous administration of the test compound. Arterial blood pressure is monitored by telemetry in freely moving, conscious animals.

Cynomolgus Monkey: Six male naïve cynomolgus monkeys weighing between 2.5 and 3.5 kg can be used in the studies. At least 4 weeks before the experiment, the monkeys are anesthetized with ketamine hydrochloride (15 mg/kg, i.m.) and xylazine hydrochloride (0.7 mg/kg, i.m.), and are implanted into the abdominal cavity with a transmitter (Model #TL11M2-D70-PCT, Data Sciences, St. Paul, Minn.). The pressure catheter is inserted into the lower abdominal aorta via the femoral artery. The bipotential leads are placed in Lead II configuration. The animals are housed under constant temperature (19-25° C.), humidity (>40%) and lighting conditions (12 h light and dark cycle), are fed once daily, and are allowed free access to water. The animals are sodium depleted by placing them on a low sodium diet (0.026%, Expanded Primate Diet 829552 MP-VENaCl (P), Special Diet Services, Ltd., UK) 7 days before the experiment and furosemide (3 mg/kg, intramuscularly i.m., Aventis Pharmaceuticals) is administered at −40 h and −16 h prior to administration of test compound.

For oral dosing, the renin inhibitors are formulated in 0.5% methylcellulose at dose levels of 10 and 30 mg/kg (5 mL/kg) by infant feeding tubes. For intravenous delivery, a silastic catheter is implanted into posterior vena cava via a femoral vein. The catheter is attached to the delivery pump via a tether system and a swivel joint. Test compound (dose levels of 0.1 to 10 mg/kg, formulated at 5% dextrose) is administered by continuous infusion (1.67 mL/kg/h) or by bolus injection (3.33 mL/kg in 2 min).

Arterial blood pressures (systolic, diastolic and mean) and body temperature are recorded continuously at 500 Hz and 50 Hz, respectively, using the Dataquest™ A.R.T. (Advanced Research Technology) software. Heart rate is derived from the phasic blood pressure tracing. During the recording period, the monkeys are kept in a separate room without human presence to avoid pressure changes secondary to stress. All data are expressed as mean±SEM. Effects of the renin inhibitors on blood pressure are assessed by ANOVA, taking into account the factors dose and time compared with the vehicle group.

Beagle Dogs: Non-naive Beagle dogs (2 per sex) weighing between 9 and 11 kg can be used in the studies. Each animal is implanted subcutaneously with a telemetry transmitter (Data Sciences) and the blood pressure catheter is inserted into the left femoral artery. The electrocardiogram leads are also tunneled subcutaneously to the appropriate anatomical regions. The animals are housed under constant temperature and lighting conditions, are fed once daily, and are allowed free access to water. A sodium depleted state is produced by placing them on a low-sodium diet (<4 meq/day, a combination of canned Prescription Diet canine h/d, from Hill's Pet Products and dry pellets from Bio-Serv Inc., Frenchtown, N.J.) beginning 10 days before the experiment, and furosemide (3 mg/kg i.m.; Aventis Pharmaceuticals) is administered at −40 and −16 h prior to administration of test compound.

A renin inhibitor is orally administered by orogastric gavage to all overnight fasted animals at a dose level of 30 mg/kg (4 mL/kg formulated in 0.5% methylcellulose). Food is given 4 h postdose. In some experiments, the renin inhibitor is administered by bolus i.v. at increasing dose levels of 1, 3 and 6 mg/kg (2, 6 and 20 mg/mL formulated in sterile saline). Cardiovascular parameters are collected continuously at least 80 min predose and 3 h postdose, followed by every 10 min for 5 h and every 30 min for 16 h postdose. The Dataquest™ ART (version 2.2) software package from DSI (Data Sciences International) is used to collect telemetered cardiovascular data.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A compound represented by the following Structural Formula: 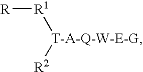
or an enantiomer, a diastereomer or a pharmaceutically acceptable salt thereof, wherein:
R is:
a) hydrogen;
b) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cycloalkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidine, pyrrolidino, (C₃-C₇)cycloalkylamino, ((C₃-C₇)cycloalkyl(C₁-C₃)alkyl)amino, or tri(C₁-C₄)alkylsilyl, each optionally and independently substituted with zero to four substituents selected from the group consisting of halogen, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;
c) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃)alkenyl, or heteroaryl(C₂-C₃)alkynyl, each optionally and independently substituted with zero to three substituents selected from the group consisting of: halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or
d) a divalent radical selected from —(CH₂)₃—, —(CH₂)₄—, —(CH₂)₅— or —(CH₂)₆—, which is attached to R¹ to form a fused or spirofused ring system, and is optionally and independently substituted with zero to four substituents selected from: halogen, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally and independently substituted with zero to four substituents selected from: halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkythio, (C₄-C₇)cycloalkylalkylthio, halo(C₁-C₆)alkylthio, halo(C₃-C₆)cycloalkythio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl;
R² is:
a) —H; or
b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxycarbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkyl, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonylamino(C₁-C₆)alkyl, (C₁-C₆)alkylaminocarbonylamino(C₁-C₆)alkoxy, (C₁-C₆)alkylaminocarbonylamino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkyl aminocarbonylamino, or (C₁-C₁₂)alkanoylamino, each optionally substituted by:
1) 1 to 5 halogen atoms; and/or
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
wherein the divalent sulfur atoms are optionally and independently oxidized to sulfoxide or sulfone, and wherein the carbonyl groups are optionally and independently changed to a thiocarbonyl groups;
T is N or CR³;
R³ is hydrogen, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonylamino, phenylamino or heteroarylamino in which each phenylamino or heteroarylamino group is optionally substituted with 1 to 5 groups independently selected from the group consisting of halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, and di(C₁-C₆)alkylaminocarbonyl;
provided that:
R² and R³ are not both hydrogen; and
ii) when T is N or T is CR³ and R³ is hydroxy, halogen, or optionally substituted phenylamino or heteroarylamino, R² is not an optionally substituted alkoxy, alkylthio or amino group as follows: (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonylamino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl(C₁-C₆)alkylamino, (C₁-C₆) acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkoxy, aminosulfonylamino(C₁-C₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonylamino(C₁-C₆)alkoxy, (C₁-C₆)alkylaminocarbonylamino(C₁-C₆)alkylthio, (C₁-C₆)alkylaminocarbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylaminocarbonylamino, or (C₁-C₁₂)alkanoylamino, each optionally substituted by:
1) 1 to 5 halogen atoms; and/or
2) 1 group selected from cyano, hydroxy, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, or halo(C₃-C₆)cycloalkoxy;
wherein the divalent sulfur atoms are optionally and independently oxidized to sulfoxide or sulfone, and wherein the carbonyl groups are optionally and independently changed to thiocarbonyl groups;
A is represented by the following Structural Formula: 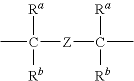
R^{a} and R^{b}, for each occurrence, are independently hydrogen, (C₁-C₆)alkyl, or halo(C₁-C₆)alkyl or R^{a} and R^{b} attached to one carbon atom taken together are an oxo;
Z is —O—, —S—, —(NR⁷)— or —(CR^{a}R^{b})—, wherein R⁷ is (C₁-C₆)alkyl, halo(C₁-C₆)alkyl or (C₁-C₃)alkoxy(C₁-C₃)alkyl;
m and n are 0, 1 or 2 and m+n=2; provided when T is N and Z is —O—, —S— or —NR⁷—, then n is 2;
Q is a divalent radical selected from 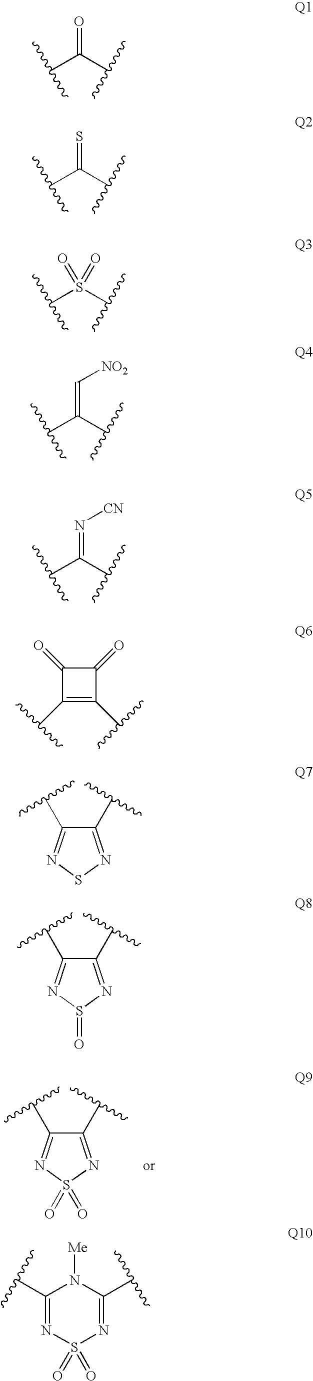
wherein A and W or E are attached to the truncated bonds
W is a bond or a (C₁-C₆)alkylene; and
W is optionally and independently substituted by zero to four groups selected from:
1) (C₁-C₁₂)alkyl, (C₃-C₈)cycloalkyl, (C₃-C₈)cycloalkyl(C₁-C₃)alkyl, (C₂-C₁₂)alkenyl, (C₅-C₈)cycloalkyl(C₁-C₃)alkenyl, (C₂-C₁₂)alkynyl, (C₃-C₈)cycloalkyl(C₁-C₃)alkynyl, (C₄-C₁₂)bicycloalkyl(C₁-C₃)alkyl, (C₈-C₁₄)tricycloalkyl(C₁-C₃)alkyl, (C₁-C₆)alkoxy(C₁-C₆)alkyl, (C₃-C₈)cycloalkoxy(C₁-C₃)alkyl, (C₁-C₆)alkylthio(C₁-C₆)alkyl, (C₃-C₈)cycloalkylthio(C₁-C₃)alkyl, saturated heterocyclyl, saturated heterocyclyl(C₁-C₃)alkyl, hydroxy and oxo, wherein each of these groups is optionally and independently substituted by zero to six groups selected from: halogen, cyano, hydroxy, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, halo(C₃-C₆)cycloalkoxy and wherein divalent sulfur atoms are optionally oxidized to sulfoxide or sulfone; or
2) phenyl, naphthyl, heteroaryl, phenyl(C₁-C₃)alkyl, naphthyl(C₁-C₃)alkyl, and heteroaryl(C₁-C₃)alkyl, each optionally and independently substituted with zero to three groups selected from: halogen, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl-(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, aminocarbonyl, (C₁-C₆)alkylaminocarbonyl, di(C₁-C₆)alkylaminocarbonyl, cyano(C₁-C₆)alkyl, hydroxy(C₁-C₆)alkyl, carboxy(C₁-C₆)alkyl, (C₁-C₆)alkoxy(C₁-C₆)alkyl, (C₃-C₈)cycloalkoxy(C₁-C₆)alkyl, (C₄-C₈)cycloalkylalkoxy(C₁-C₆)alkyl, halo(C₁-C₆)alkoxy(C₁-C₆)alkyl, halo(C₃-C₆)cycloalkoxy(C₁-C₆)alkyl, halo(C₄-C₈)cycloalkylalkoxy(C₁-C₆)alkyl, (C₁-C₈)alkylthio(C₁-C₆)alkyl, (C₃-C₈)cycloalkylthio(C₁-C₆)alkyl, (C₄-C₈)cycloalkylalkylthio(C₁-C₆)alkyl, halo(C₁-C₈)alkylthio(C₁-C₆)alkyl, halo(C₃-C₈)cycloalkylthio(C₁-C₆)alkyl, halo(C₄-C₈)cycloalkylalkylthio(C₁-C₆)alkyl, (C₁-C₈)alkanesulfinyl(C₁-C₆)alkyl, (C₃-C₈)cycloalkanesulfinyl(C₁-C₆)alkyl, (C₄-C₈)cycloalkylalkanesulfinyl(C₁-C₆)alkyl, halo(C₁-C₈)alkanesulfinyl(C₁-C₆)alkyl, halo(C₃-C₈)cycloalkanesulfinyl(C₁-C₆)alkyl, halo(C₄-C₈)cycloalkylalkanesulfinyl(C₁-C₆)alkyl, (C₁-C₈)alkanesulfonyl(C₁-C₆)alkyl, (C₃-C₈)cycloalkanesulfonyl(C₁-C₆)alkyl, (C₄-C₈)cycloalkylalkanesulfonyl(C₁-C₆)alkyl, halo(C₁-C₈)alkanesulfonyl(C₁-C₆)alkyl, halo(C₃-C₈)cycloalkanesulfonyl(C₁-C₆)alkyl, halo(C₄-C₈)cycloalkylalkanesulfonyl(C₁-C₆)alkyl, (C₁-C₈)alkylamino(C₁-C₆)alkyl, di(C₁-C₈)alkylamino(C₁-C₆)alkyl, (C₁-C₈)alkoxycarbonyl(C₁-C₆)alkyl, (C₁-C₈)acyloxy(C₁-C₆)alkyl, aminocarbonyl(C₁-C₆)alkyl, (C₁-C₈)alkylaminocarbonyl(C₁-C₆)alkyl, di(C₁-C₈)alkylaminocarbonyl(C₁-C₆)alkyl(C₁-C₈)acylamino(C₁-C₆)alkyl, (C₁-C₈)alkoxycarbonylamino, (C₁-C₈)alkoxycarbonylamino(C₁-C₆)alkyl, aminocarboxy(C₁-C₆)alkyl, (C₁-C₈)alkylaminocarboxy(C₁-C₆)alkyl and di(C₁-C₈)alkylaminocarboxy(C₁-C₆)alkyl, phenyl, naphthyl, heteroaryl, bicyclic heteroaryl, phenoxy, naphthyloxy, heteroaryloxy, bicyclic heteroaryloxy, phenylthio, naphthylthio, heteroarylthio, bicyclic heteroarylthio, phenylsulfinyl, naphthylsulfinyl, heteroarylsulfinyl, bicyclic heteroarylsulfinyl, phenylsulfonyl, naphthylsulfonyl, heteroarylsulfonyl, bicyclic heteroarylsulfonyl, phenyl(C₁-C₃)alkyl, naphthyl(C₁-C₃)alkyl, heteroaryl(C₁-C₃)alkyl, and bicyclic heteroaryl(C₁-C₃)alkyl, wherein the aromatic and heteroaromatic groups are optionally and independently substituted with zero to three groups selected from: halogen, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;
E is a saturated or unsaturated 3-, 4-, 5-, 6-, or 7-membered ring which is optionally bridged by (CH₂)ₙ via bonds to two members of said ring, wherein said ring is composed of carbon atoms and zero to four hetero atoms selected from: zero to four nitrogen atoms, zero or one oxygen atoms, and zero or one sulfur atoms, said ring being optionally and independently substituted with zero to four groups selected from: halogen, hydroxy, (C₁-C₆)alkyl, (C₃-C₈)cycloalkyl(C₁-C₆)alkyl, halo(C₁-C₆)alkyl, hydroxy(C₁-C₆)alkyl, and oxo groups, such that when there is substitution with one oxo group on a carbon atom it forms a carbonyl group, and when there is substitution of one or two oxo groups on sulfur it forms sulfoxide or sulfone groups, respectively;
n is 1 to 3;
G is hydrogen, (C₁-C₆)alkyl, (C₄-C₇)heterocyclyl, hydroxy, hydroxy(C₁-C₆)alkyl, —NR^{4a}R⁴, —O(C₁-C₆)alkyl-NR^{4a}R⁴, amino(C₁-C₆)alkylcarboxy, (C₃-C₈)cycloalkyl, (C₁-C₆)alkylamino(C₁-C₆)alkyl, amino(C₁-C₆)alkyl, (C₁-C₆)alkylamino, di(C₁-C₆)alkylamino, di(C₁-C₆)alkylamino(C₁-C₆)alkyl, C(═NH)NH₂, C(═NH)NHR⁴, NHC(═NH)NH₂, NHC(═NH)NHR⁴; —(C₀-C₆)alkyl-NR⁴R^{4a}, —NHC(═NH)NR⁴R^{4a}, —C(═O)(C₁-C₆)alkyl-NR⁴R^{4a}, —C(═NH)NR⁴R^{4a}, —C(═O)(C₁-C₄)alkylaryl,
—C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino;
and where R^{4a} is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;
provided that when T is N, E is not 1,2-cyclopentylene.

2. The compound of claim 1, wherein the compound is represented by the following Structural Formula: 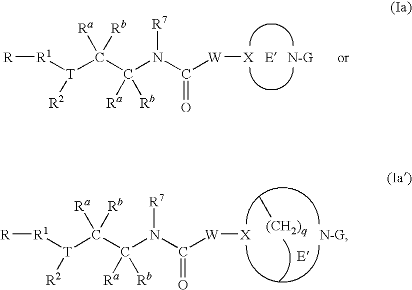
or an enantiomer, a diastereomer or a pharmaceutically acceptable salt thereof, wherein:
ring E′ is a saturated 4-, 5-, 6-, or 7-membered heterocyclic ring which is optionally bridged by (CH₂)_{q} via bonds to two members of said ring; wherein ring E′ is optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;
X is a ring carbon atom or nitrogen atom bonded directly to W;
W is a bond or a (C₁-C₃)alkyl; and
q is 1 to 3.

3. The compound of claim 2, wherein ring E′ is selected from the group consisting of piperidinyl, piperazinyl, and pyrrolidinyl, said group being optionally substituted with a hydroxy, (C₁-C₃)alkyl or halo(C₁-C₃)alkyl group.

4. The compound of claim 3, wherein G is hydrogen, (C₁-C₆)alkyl, heterocyclyl, —(C₂-C₆)alkyl-OH, —(C₂-C₆)alkyl-NR^{4a}, —C(═O)(C₁-C₆)alkyl-NR⁴R^{4a}, —C(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino.

5. The compound of claim 4, wherein G is hydrogen, heterocyclyl, —(C₂-C₄)alkyl-OH, —(C₂-C₄)alkyl-NR⁴R^{4a}, —C(═O)(C₁-C₄)alkyl-NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₇)cycloalkyl, or —(C₁-C₄)alkyl(C₄-C₇)heterocyclyl.

6. The compound of claim 1, wherein the compound is represented by the following Structural Formula: 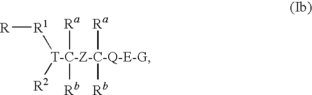
or an enantiomer, a diastereomer or a pharmaceutically acceptable salt thereof.

7. The compound of claim 6, wherein the compound is represented by the following Structural Formula: 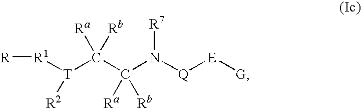
or an enantiomer, a diastereomer or a pharmaceutically acceptable salt thereof.

8. The compound of claim 7, wherein the compound is represented by the following Structural Formula: 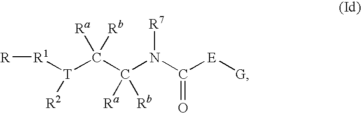
or an enantiomer, a diastereomer or a pharmaceutically acceptable salt thereof.

9. The compound of claim 8, wherein the compound is represented by the following Structural Formula: 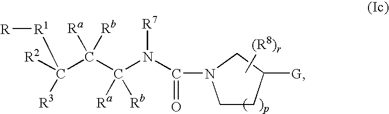
or an enantiomer, a diastereomer or a pharmaceutically acceptable salt thereof, wherein:
p is 1 or 2; R⁸ is selected from the group consisting of halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group; and r is 0, 1 or 2 when p is 1 or r is 0, 1, 2 or 3 when p is 2.

10. The compound of claim 9, wherein G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl

11. The compound of claim 8, wherein the compound is represented by the following Structural Formula: 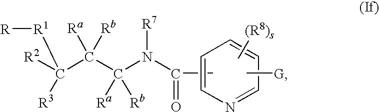
or an enantiomer, a diastereomer or a pharmaceutically acceptable salt thereof, wherein:
s is 0, 1, 2, 3 or 4 and R⁸ is selected from the group consisting of halogen, hydroxy, (C₁-C₆)alkyl, halo(C₁-C₆)alkyl, hydroxy(C₁-C₆)alkyl and oxo groups such that when there is substitution with one oxo group on a carbon atom it forms a carbonyl group.

12. The compound of claim 11, wherein G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl.

13. The compound of claim 8, wherein the compound is represented by the following Structural Formula: 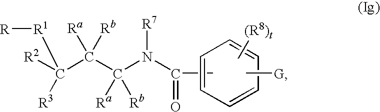
or an enantiomer, a diastereomer or a pharmaceutically acceptable salt thereof, wherein:
t is 0, 1, 2, 3 or 4 and R⁸ is selected from the group consisting of halogen, hydroxy, (C₁-C₆)alkyl, halo(C₁-C₆)alkyl, hydroxy(C₁-C₆)alkyl and oxo groups such that when there is substitution with one oxo group on a carbon atom it forms a carbonyl group.

14. The compound of claim 13, wherein G is hydroxy, —NR⁴R^{4a}, —O(C₂-C₆)alkyl-NR⁴R^{4a}, heterocyclyl, —(C₁-C₆)alkyl-OH, —(C₁-C₆)alkyl-NR⁴R^{4a}, —C(═O)(C₁-C₆)alkyl-NR⁴R^{4a}, NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino.

15. The compound of claim 8, wherein the compound is represented by the following Structural Formula: 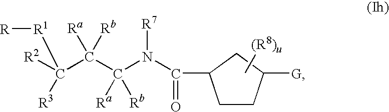
or an enantiomer, a diastereomer or a pharmaceutically acceptable salt thereof, wherein:
u is 0, 1, 2 or 3; and R⁸ halogen, hydroxy, (C₁-C₆)alkyl, halo(C₁-C₆)alkyl, hydroxy(C₁-C₆)alkyl and oxo groups such that when there is substitution with one oxo group on a carbon atom it forms a carbonyl group.

16. The compound of claim 15, wherein G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl.

17. The compound of claim 2, wherein:
R is a) (C₁-C₅)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or
b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: halogen, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or
c) a divalent radical selected from —(CH₂)₄— or —(CH₂)₅—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, or (C₃-C₇)cycloalkyl ring optionally substituted with up to four substituents independently selected from the group consisting of fluorine, chlorine, bromine, cyano, (C₁-C₆)alkyl, (C₃-C₆)cycloalkyl, halo(C₁-C₆)allyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl;
R² is a) —H; or b) (C₁-C₁₀)alkyl, (C₂-C₁₀)alkenyl, (C₂-C₁₀)alkynyl, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkyl, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁*C₅)alkyamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted by
1) 1 to 5 fluorine atoms; and/or
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy; wherein the divalent sulfur atoms are independently optionally oxidized to sulfoxide or sulfone; and
R³ is —H, halogen, (C₁-C₃)alkyl, (C₁-C₃)alkoxy, hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylaminocarbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from the group consisting of fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;
provided that
i) R² and R³ are not both hydrogen and
ii) when T is N or T is CR³ and R³ is hydroxyl, halogen, or optionally substituted phenylamino or heteroarylamino, R² is not an optionally substituted alkoxy, alkylthio or amino group as follows: (C₁-C₁₀)alkoxy, (C₁-C₁₀)alkylthio, (C₁-C₁₀)alkylamino, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, aminocarbonylamino(C₁-C₁₀)alkoxy, aminocarbonylamino(C₁-C₁₀)alkylthio, aminocarbonyl-amino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted with
1) 1 to 5 fluorine atoms; and/or
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy;
wherein the divalent sulfur atoms are independently optionally oxidized to sulfoxide or sulfone.

18. The compound of claim 17, wherein:
R is a) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from the group consisting of fluorine, hydroxy, (C₁-C₃)alkyl, and halo(C₁-C₃)alkyl,
b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from the group consisting of halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or
c) a divalent radical selected from —(CH₂)₄— or —(CH₂)₅—, which is attached to R¹ to form a fused or spirofused ring system;
R¹ is phenyl, monocyclic heteroaryl ring, bicyclic heteroaryl ring or benzo-1,3-dioxole, optionally substituted with up to four substituents independently selected from the group consisting of halogen, cyano, (C₁-C₃)alkyl, (C₃-C₄)cycloalkyl, halo(C₁-C₃)alkyl, (C₁-C₃)alkoxy, halo(C₁-C₃)alkoxy, and H₂NCO;
R² is —H, (C₁-C₈)alkyl, (C₄-C₉)cycloalkylalkyl, fluoro(C₁-C₈)alkyl, fluoro(C₄-C₉)-cycloalkyl alkyl, (C₁-C₈)alkoxy, (C₄-C₉)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)-cycloalkanecarbonyllamino(C₁-C₅)alkyl, (C₃-C₄)cycloalkanecarbonyllamino(C₁-C₅)alkoxy, aminosulfonylamino(C₁-C₈)alkyl, aminosulfonylamino(C₁-C₈)alkoxy, (C₁-C₅)alkane-sulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylamino-carbonylamino(C₁C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
R³ is —H, halogen, OH, (C₁-C₄)alkanoylamino, or (C₁-C₃)alkoxy;
provided that
R² and R³ are not both hydrogen; and
ii) when T is N or T is CR³ and R³ is OH or halogen, R² is not an optionally substituted alkoxy, alkylthio or amino group as follows: (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)-alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkanecarbonyllamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxy-carbonylamino, (C₁-C₈)alkylaminocarbonylamino, (C₁-C₈)alkanoylamino, fluoro(C₁-C₈)alkoxy-carbonylamino, fluoro(C₁-C₈)alkylaminocarbonylamino, or fluoro(C₁-C₈)alkanoylamino.

19. The compound of claim 18, wherein:
R is a) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, or halo(C₁-C₃)alkyl; or
b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio or H₂NCO; or
c) —(CH₂)₄— or —(CH₂)₅—, which is attached to R¹ to form a fused or spirofused ring system;
R¹ is phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—;
R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₄)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl, (C₁-C₃)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy; and
R³ is hydrogen, fluoro, hydroxyl, or (C₁-C₄)alkanoylamino, provided that when T is N or T is CR³ and R³ is hydroxyl or fluoro, R² is not (C₁-C₃)alkoxy(C₁-C₅)alkoxy, (C₃-C₄)cycloalkyl(C₁-C₅)alkoxy, (C₁-C₃)alkoxy-carbonylamino(C₁-C₅)alkoxy, (C₁-C₃)alkanoylamino(C₁-C₅)alkoxy or (C₁-C₃)alkylaminocarbonyl(C₁-C₅)alkoxy.

20. The compound of claim 19, wherein G is —NHR⁹ or (C₁-C₃)alkyl-NHR⁹, wherein R⁹ is H or (C₁-C₆)alkyl.

21. The compound of claim 20, wherein R² is (C₁-C₃)alkoxy(C₁-C₅)alkyl, (C₁-C₃)alkoxy(C₁-C₅)alkoxy, (C₁-C₃)alkoxycarbonylamino(C₁-C₅)alkyl, (C₁-C₃)-alkoxycarbonylamino(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, or (C₁-C₅)alkylamino-carboxy(C₁-C₅)alkoxy.

22. The compound of claim 21, wherein:
R¹ is phenyl optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—; and
R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—, which is attached to R¹ to form a fused or spirofused ring system.

23. The compound of claim 22, wherein:
R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl; and
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole; and
R² is 4-methoxybutyl, 4-ethoxybutyl, 4-methoxypentyl, 3-methoxypropoxy, 3-(methoxycarbonylamino)propyl, or 2-(methoxycarbonylamino)ethoxy.

24. The compound of claim 23, wherein R³ is hydrogen, hydroxyl or methoxycarbonylamino, provided that when R³ is hydroxyl, R² is not 3-methoxypropoxy, 2-(acetylamino)ethoxy, or 2-(methoxycarbonylamino)ethoxy.

25. The compound of claim 24, wherein:
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl
R⁷ is methyl, ethyl, propyl, or isopropyl;
R^{a} and R^{b}, for each occurrence, are independently H, methyl or R^{a} and R^{b} attached to one carbon atom taken together are an oxo; and
G is —NHR⁹ or CH₂NHR⁹, wherein R⁹ is H, methyl or ethyl.

26. The compound of claim 25, wherein:
R is phenyl, 3-methylphenyl or 3-ethylphenyl;
R¹ is phenyl or 3-chlorophenyl;
R² is 4-methoxybutyl;
R³ is hydroxyl;
R⁷ is methyl; and
G is —NH₂ or —CH₂NH₂.

27. The compound of claim 19, wherein R² is 3-(acetylamino)propyl or 2-(acetylamino)ethoxy.

28. The compound of claim 27, wherein G is —NHR⁹ or (C₁-C₃)alkyl-NHR⁹, wherein R⁹ is H or (C₁-C₆)alkyl.

29. The compound of claim 28, wherein:
R¹ is phenyl optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—; and
R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—, which is attached to R¹ to form a fused or spirofused ring system.

30. The compound of claim 29, wherein:
R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl; and
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole.

31. The compound of claim 30, wherein R³ is hydrogen, hydroxyl or methoxycarbonylamino, provided that when R³ is hydroxyl, R² is not 2-(acetylamino)ethoxy.

32. The compound of claim 31, wherein:
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl
R⁷ is methyl, ethyl, propyl, or isopropyl;
R^{a} and R^{b}, for each occurrence, are independently H, methyl or R^{a} and R^{b} attached to one carbon atom taken together are an oxo; and
G is —NHR⁹ or CH₂NHR⁹, wherein R⁹ is H, methyl or ethyl.

33. The compound of claim 32, wherein:
R is phenyl, 3-methylphenyl or 3-ethylphenyl;
R¹ is phenyl or 3-chlorophenyl;
R³ is hydroxyl;
R⁷ is methyl; and
G is —NH₂ or —CH₂NH₂.

34. The compound of claim 8, wherein the compound is represented by the following Structural Formula: 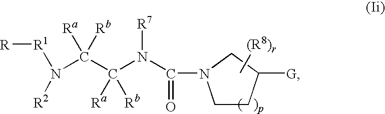
or an enantiomer, a diastereomer, or a pharmaceutically acceptable salt thereof, wherein:
p is 1 or 2; R⁸ is selected from the group consisting of halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group; and r is 0, 1 or 2 when p is 1 or r is 0, 1, 2 or 3 when p is 2.

35. The compound of claim 34, wherein G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl

36. The compound of claim 8, wherein the compound is represented by the following Structural Formula: 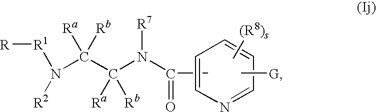
or an enantiomer, a diastereomer, or a pharmaceutically acceptable salt thereof, wherein:
s is 0, 1, 2, 3 or 4 and R⁸ is selected from the group consisting of halogen, hydroxy, (C₁-C₆)alkyl, halo(C₁-C₆)alkyl, hydroxy(C₁-C₆)alkyl and oxo groups such that when there is substitution with one oxo group on a carbon atom it forms a carbonyl group.

37. The compound of claim 36, wherein G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl.

38. The compound of claim 8, wherein the compound is represented by the following Structural Formula: 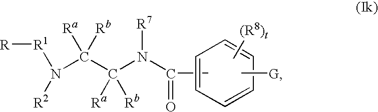
or an enantiomer, a diastereomer, or a pharmaceutically acceptable salt thereof, wherein:
t is 0, 1, 2, 3 or 4 and R⁸ is selected from the group consisting of halogen, hydroxy, (C₁-C₆)alkyl, halo(C₁-C₆)alkyl, hydroxy(C₁-C₆)alkyl and oxo groups such that when there is substitution with one oxo group on a carbon atom it forms a carbonyl group.

39. The compound of claim 38, wherein G is hydroxy, —NR⁴R^{4a}, —O(C₂-C₆)alkyl-NR⁴R^{4a}, heterocyclyl, —(C₁-C₆)alkyl-OH, —(C₁-C₆)alkyl-NR⁴R^{4a}, —C(═O)(C₁-C₆)alkyl-NR⁴R^{4a}, —C(═NH)NR⁴R^{4a}, —NHC(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino.

40. The compound of claim 8, wherein the compound is represented by the following Structural Formula: 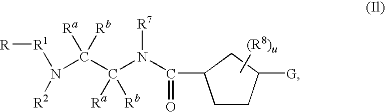
or an enantiomer, a diastereomer, or a pharmaceutically acceptable salt thereof, wherein:
u is 0, 1, 2 or 3; and R⁸ is selected from the group consisting of halogen, hydroxy, (C₁-C₆)alkyl, halo(C₃-C₆)alkyl, hydroxy(C₁-C₆)alkyl and oxo groups such that when there is substitution with one oxo group on a carbon atom it forms a carbonyl group.

41. The compound of claim 40, wherein G is hydroxy, hydroxy(C₁-C₆)alkyl, —NR^{4a}R⁴, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; wherein R⁴ is (C₁-C₃)alkyl.

42. The compound of claim 36, wherein:
R is a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₈)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or
b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: halogen, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or
c) a divalent radical selected from —(CH₂)₄— or —(CH₂)₅—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, or (C₃-C₇)cycloalkyl ring optionally substituted with up to four substituents independently selected from the group consisting of fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl; and
R² is a) —H; or b) (C₁-C₁₀)alkyl, (C₂-C₁₀)alkenyl, (C₂-C₁₀)alkynyl, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₃)alkoxy(C₁-C₃)alkoxy(C₁-C₃)alkyl, aminocarbonylamino(C₁-C₁₀)alkyl, (C₁-C₅)alkanoylamino(C₁-C₅)alkyl, aminosulfonylamino(C₁-C₁₀)alkyl, (C₁-C₅)alkanesulfonylamino(C₁-C₅)alkyl, formylamino(C₁-C₅)alkyl, (C₁-C₅)alkoxycarbonylamino(C₁-C₅)alkyl, (C₁-C₅)alkylaminocarbonylamino(C₁-C₅)alkyl, aminocarbonyl(C₁-C₅)alkyl, (C₁-C₅)alkylaminocarbonyl(C₁-C₅)alkyl, aminocarboxy(C₁-C₅)alkyl, (C₁-C₅)alkylaminocarboxy(C₁-C₅)alkyl, each optionally substituted by
1) 1 to 5 fluorine atoms; and/or
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy; wherein the divalent sulfur atoms are independently optionally oxidized to sulfoxide or sulfone.

43. The compound of claim 42, wherein:
R is a) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from the group consisting of fluorine, hydroxy, (C₁-C₃)alkyl, and halo(C₁-C₃)alkyl,
b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from the group consisting of halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or
c) a divalent radical selected from —(CH₂)₄— or —(CH₂)₅—, which is attached to R¹ to form a fused or spirofused ring system;
R¹ is phenyl, monocyclic heteroaryl ring, bicyclic heteroaryl ring or benzo-1,3-dioxole, optionally substituted with up to four substituents independently selected from the group consisting of halogen, cyano, (C₁-C₃)alkyl, (C₃-C₄)cycloalkyl, halo(C₁-C₃)alkyl, (C₁-C₃)alkoxy, halo(C₁-C₃)alkoxy, and H₂NCO;
R² is —H, (C₁-C₈)alkyl, (C₄-C₉)cycloalkylalkyl, fluoro(C₁-C₈)alkyl, fluoro(C₄-C₉)-cycloalkyl alkyl, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, (C₁-C₅)alkanoylamino(C₁-C₅)alkyl, fluoro(C₁-C₅)alkanoylamino(C₁-C₅)alkyl, (C₁-C₃)alkoxy(C₁-C₅)alkanoylamino(C₁-C₅)alkyl, (C₃-C₄)-cycloalkanecarbonyllamino(C₁-C₅)alkyl, aminosulfonylamino(C₁-C₈)alkyl, (C₁-C₅)alkane-sulfonylamino(C₁-C₅)alkyl, formylamino(C₁-C₅)alkyl, (C₁-C₅)alkoxycarbonylamino(C₁-C₅)alkyl, (C₁-C₅)alkylaminocarbonylamino(C₁-C₅)alkyl, (C₁-C₅)alkylamino-carbonylamino(C₁-C₅)alkyl, aminocarbonyl(C₁-C₅)alkyl, aminocarbonyl(C₁-C₅)alkoxy, (C₁-C₅)alkylaminocarbonyl(C₁-C₅)alkyl, aminocarboxy(C₁-C₅)alkyl, or (C₁-C₅)alkylaminocarboxy(C₁-C₅)alkyl.

44. The compound of claim 43, wherein:
R is a) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, or halo(C₁-C₃)alkyl; or
b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio or H₂NCO; or
c) —(CH₂)₄— or —(CH₂)₅—, which is attached to R¹ to form a fused or spirofused ring system;
R¹ is phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—; and
R² is (C₁-C₃)alkoxy(C₁-C₅)alkyl, (C₃-C₄)cycloalkyl(C₁-C₅)alkyl, (C₁-C₃)alkoxycarbonylamino(C₁-C₅)alkyl, (C₁-C₃)alkanoylamino(C₁-C₅)alkyl, (C₁-C₃)alkylaminocarbonyl(C₁-C₅)alkyl, aminocarboxy(C₁-C₅)alkyl, or (C₁-C₅)alkylaminocarboxy(C₁-C₅)alkyl.

45. The compound of claim 44, wherein G is —NHR⁹ or (C₁-C₃)alkyl-NHR⁹, wherein R⁹ is H or (C₁-C₆)alkyl.

46. The compound of claim 45, wherein R² is (C₁-C₃)alkoxy(C₁-C₅)alkyl, (C₁-C₃)alkoxycarbonylamino(C₁-C₅)alkyl, aminocarboxy(C₁-C₅)alkyl, or (C₁-C₅)alkylaminocarboxy(C₁-C₅)alkyl.

47. The compound of claim 46, wherein:
R¹ is phenyl optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—; and
R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropyl phenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—, which is attached to R¹ to form a fused or spirofused ring system.

48. The compound of claim 47, wherein:
R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl; and
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole; and
R² is 4-methoxybutyl, 4-ethoxybutyl, 4-methoxypentyl, or 3-(methoxycarbonylamino)propyl.

49. The compound of claim 48, wherein:
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl
R⁷ is methyl, ethyl, propyl, or isopropyl;
R^{a} and R^{b}, for each occurrence, are independently H, methyl or R^{a} and R^{b} attached to one carbon atom taken together are an oxo; and
G is —NHR⁹ or —CH₂NHR⁹, wherein R⁹ is H, methyl or ethyl.

50. The compound of claim 49, wherein:
R is phenyl, 3-methylphenyl or 3-ethylphenyl;
R¹ is phenyl or 3-chlorophenyl;
R⁷ is methyl; and
G is —NH₂ or —CH₂NH₂.

51. The compound of claim 44, wherein R² is 3-(acetylamino)propyl.

52. The compound of claim 51, wherein G is —NHR⁹ or (C₁-C₃)alkyl-NHR⁹, wherein R⁹ is H or (C₁-C₆)alkyl.

53. The compound of claim 52, wherein:
R¹ is phenyl optionally substituted with up to 3 substituents independently selected from the group consisting of fluorine, chlorine, cyano, (C₁-C₃)alkyl, halo(C₁-C₃)alkyl, (C₁-C₃)alkoxy, and H₂NCO—;
R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropyl phenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅.

54. The compound of claim 53, wherein:
R is phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, or 3,5-dimethylphenyl; and
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole.

55. The compound of claim 54, wherein:
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl
R⁷ is methyl, ethyl, propyl, or isopropyl;
R^{a} and R^{b}, for each occurrence, are independently H, methyl or R^{a} and R^{b} attached to one carbon atom taken together are an oxo; and
G is —NHR⁹ or —CH₂NHR⁹, wherein R⁹ is H, methyl or ethyl.

56. The compound of claim 55, wherein:
R is phenyl, 3-methylphenyl or 3-ethylphenyl;
R¹ is phenyl or 3-chlorophenyl;
R⁷ is methyl; and
G is —NH₂ or CH₂NH₂.

57. The compound of claim 1, wherein the compound is selected from the group consisting of:
(1S,3R,4S)-3-amino-N-[3-(6-chloro-3′-methyl-2-biphenylyl)-3-hydroxy-7-(methyloxy)heptyl]-4-hydroxy-N-methylcyclopentanecarboxamide;
(1R,3S)-3-amino-N-[3-(6-chloro-3′-ethyl-2-biphenylyl)-3-hydroxy-7-(methyloxy)heptyl]-N-methylcyclopentanecarboxamide;
(1S,3R,4S)-3-amino-N-[3-(6-chloro-3¹-ethyl-2-biphenylyl)-3-hydroxy-7-(methyloxy)heptyl]-4-hydroxy-N-methylcyclopentanecarboxamide;
4-(aminomethyl)-N-[3-(6-chloro-3′-ethyl-2-biphenylyl)-3-hydroxy-7-(methyloxy)heptyl]-N-methylbenzamide;
(1R,3S)-3-amino-N-(2-{(6-chloro-3′-ethyl-2-biphenylyl)[4-(methyloxy)butyl]amino}ethyl)-N-methylcyclopentanecarboxamide;
(1S,3R,4S)-3-amino-N-(2-{(6-chloro-3′-ethyl-2-biphenylyl)[4-(methyloxy)butyl]amino}ethyl)-4-hydroxy-N-methylcyclopentanecarboxamide;
(1R,3S)-3-amino-N-(2-{(6-chloro-3′-ethyl-2-biphenyl)[4-(methyloxy)butyl]amino}-2-oxoethyl)-N-methylcyclopentanecarboxamide;
(1S,3R,4S)-3-amino-N-(2-{(6-chloro-3′-ethyl-2-biphenylyl)[4-(methyloxy)butyl]amino}-2-oxoethyl)-4-hydroxy-N-methylcyclopentanecarboxamide;
(1R,3S)-3-amino-N-(3-(6-chloro-3′-methylbiphenyl-2-yl)-3-hydroxy-7-methoxyheptyl)-N-methylcyclopentanecarboxamide;
(1S,3R,4S)—N-(3-acetamido-3-(biphenyl-2-yl)-7-methoxyheptyl)-3-amino-4-hydroxy-N-methylcyclopentanecarboxamide; and
(1S,3R,4S)-3-amino-N—((S)-3-(6-chloro-5′-methylbiphenyl-2-yl)-3-hydroxy-7-methoxyheptyl)-4-hydroxy-N-methylcyclopentanecarboxamide.

58. A pharmaceutical composition comprising a compound of claim 1, or an enantiomer, diastereomer, or salt thereof and a pharmaceutically acceptable carrier or excipient.

59. The pharmaceutical composition of claim 58, further comprising an additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

60.-70. (canceled)
